(19) United States
(12) Patent Application Publication
(10) Pub. No.: US 2011/0068290 A1
(43) Pub. Date: Mar. 24, 2011

(54) CHEMICAL MODULATION OF ELECTRONIC AND MAGNETIC PROPERTIES OF GRAPHENE

(75) Inventors: Robert C. Haddon, Riverside, CA (US); Mikhail E. Itkis, Riverside, CA (US); Palanisamy Ramesh, Riverside, CA (US); Elena Bekyarova, Riverside, CA (US); Sakhrat Khizroev, Riverside, CA (US); Jeongmin Hong, Riverside, CA (US)

(73) Assignee: THE REGENTS OF THE UNIVERSITY OF CALIFORNIA, Oakland, CA (US)

(21) Appl. No.: 12/994,678

(22) PCT Filed: May 29, 2009
(86) PCT No.: PCT/US2009/045735

§ 371 Date: Nov. 24, 2010

Related U.S. Application Data
(60) Provisional application No. 61/057,656, filed on May 30, 2008.

(51) Int. Cl. H01F 1/42; C07C 63/42; C07C 39/225; C07C 22/04; C07C 15/20; H01B 1/12; C07C 17/02; C07C 45/27; C07C 49/603; C01B 31/00; C07D 303/04; C25B 3/00; B82Y 30/00; B82Y 40/00

(52) U.S. Cl. 252/62.51 R; 562/488; 568/732; 570/128; 570/144; 585/26; 585/27; 585/454; 252/516; 570/199; 568/312; 568/326; 549/518; 549/543; 423/445 R; 205/428; 205/446; 205/455; 977/734; 977/847

(57) ABSTRACT

Compounds, compositions, systems and methods for the chemical and electrochemical modification of the electronic structure of graphene and especially epitaxial graphene (EG) are presented. Beneficially, such systems and methods allow the large-scale fabrication of electronic EG devices. Vigorous oxidative conditions may allow substantially complete removal of the EG carbon atoms and the generation of insulating regions; such processing is equivalent to that which is currently used in the semiconductor industry to lithographically etch or oxidize silicon and thereby define the physical features and electronic structure of the devices. However graphene offers an excellent opportunity for controlled modification of the hybridization of the carbon atoms from sp² to sp³ states by chemical addition of organic functional groups. We show that such chemistries offer opportunities far beyond those currently employed in the semiconductor industry for control of the local electronic structure of the graphene sheet and do not require the physical removal of areas of graphene or its oxidation, in order to generate the full complement of electronic devices necessary to produce functional electronic circuitry. Selective saturation of the π-bonds opens a band gap in the graphene electronic structure which results in a semiconducting or insulating form of graphene, while allowing the insertion of new functionality with the possibility of 3-D electronic architectures. Beneficially, these techniques allow for large-scale fabrication of electronic EG devices and integrated circuits, as they allow the generation of wires (interconnects), semiconductors (transistors), dielectrics, and insulators.

U.S. Patent Application No. 61/057,565, entitled CHEMICAL MODULATION OF ELECTRONIC PROPERTIES OF EPITAXIAL GRAPHENE, filed May 30, 2008 (Atty. Docket No. UC100.009PR) is hereby incorporated by reference in its entirety and made part of this specification.

### STATEMENT REGARDING FEDERALLY SPONSORED R&D

This invention was made with Government support under Contract Numbers H94003-06-2-0608 and H94003-07-2-0703, awarded by Center for Nanoscale Innovation for Defense (CNID), sponsored by the Department of Defense Microelectronics Activity (DOD/DMEA) and under Contract DMR-0820382, awarded by the National Science Foundation through the Materials Research Science and Engineering Center program. The Government has certain rights in this invention.

### BACKGROUND OF THE INVENTION

1. Field

Embodiments of the present invention relate to graphene and, in particular, to chemical and electrochemical modification of the electronic and magnetic structure of graphene such as epitaxial graphene.

2. Related Art

US 2004/0071624 recites processes for the chemical modification of carbon nanotubes. For example, a variety of organic compounds may be attached to the sides and ends of carbon nanotubes. Dramatized nanotubes are chemically compatible with a polymer matrix, allowing transfer of the properties of the nanotubes to the properties of the composite material as a whole.

### SUMMARY OF THE INVENTION

In embodiments described herein, a modified graphene comprises at least one sp³ orbital in the modified graphene, such as, modified epitaxial graphene. In some aspects, the modified graphene is insulating and/or semiconducting. In other aspects, the modified graphene comprises a local band gap.

In some embodiments, the modified graphene comprises at least one functional group, such as a functional group selected from the group consisting of a substituted or unsubstituted alkyl, alkenyl, alkynyl, heteroalkyl, heteroalkenyl, heteroalkynyl, alkylene, aryl, or heteroaryl group, a heteroatom, and a hydroxyl group. For example, the functional group may be phenyl, benzyl, nitrophenyl, nitrobenzyl, nap him hthalyl, dichlorocarbyl, hydroxyl, ketone, or —CF₂(CF₂)ₙCF₃, wherein n is 1-10. In some aspects, the functional group is divalent, and two carbon atoms of the graphene may be covalently bonded to the functional group.

In other aspects, the modified graphene is modified by removal of a carbon in the graphene backbone. Optionally, the modified graphene contains a heteroatom such as N or O, or halogen such as fluorine at the site of the removed carbon.

The modified graphene may be saturated to the extent to provide a modified graphene with insulating properties or may be saturated to the extent to provide a modified graphene with semiconducting properties. In some embodiments, the modified graphene has a higher resistance than pristine graphene.

In some aspects, the modified graphene is partially unsaturated, for example, at 2:18 or ˜11% coverage; and the modified graphene comprises well defined conjugated pathways and may have a lower band gap and higher mobilities than fully saturated modified graphene. In other aspects, the modified graphene is partially unsaturated, for example, at 2:8 or 25% coverage; and the partially unsaturated modified graphene comprises ill defined conjugated pathways, and may have larger band gaps than a partially unsaturated modified graphene having well defined conjugated pathways.

Embodiments of the invention are also directed to compositions comprising the modified graphene described herein. A composition may comprise a SiC substrate adjacent the modified graphene. A composition may be selected from the group consisting of an electronic component or device, a wafer, a ferromagnetic semiconductor and a field effect transistor (FET). The composition may be a wafer that comprises insulating or semiconducting regions.

Embodiments of the invention are directed to methods. A method of making the modified graphene may comprise covalently attaching a functional group to at least one carbon atom of a graphene. Methods may also comprise re-hybridizing the C-atoms in a graphene from sp² to sp³ to form a modified graphene. In some aspects the method further comprises forming semiconducting or insulating regions on the modified graphene. Methods of making a patterned graphene may comprise introducing functional groups to graphene to provide semiconducting and or insulating regions of the patterned graphene. The patterned graphene may comprise a pristine region (or other modified form of graphene), a semiconducting region, and an insulating region. Methods may also comprise functionalizing graphene to form the modified graphene described herein. In some aspects, the long-range parallel and/or anti-parallel magnetic order of the graphene samples is created at room temperature. In some aspects, the graphene comprises an A and B lattice; and the functionalizing step further comprises selectively functionalizing the A or B lattice.

The covalently attaching step referenced above may further comprise a step selected from the group consisting of adding a dichlorocarbene; spontaneous grafting of an aryl group in a solution of diazonium salts; spontaneous grafting of an aryl group with in-situ generated diazonium salt; and reacting with a radical photochemically generated from an alkyl halide. Also, the covalently attaching step may further comprise a step selected from the group consisting of electrochemically attaching an alkyl and/or aryl group to graphene by cyclic voltammetry or electrolysis of carboxylates (the Kolbe reaction); electrochemically attaching an aryl group to graphene by cyclic volammetry scans or electrolysis of a diazonium salt; electrochemically attaching an aryl and/or an alkyl group to graphene by cyclic volammetry scans or electrolysis of an aryl and/or alkyl halide; electrochemically attaching an aryl group to graphene by cyclic volammetry scans or electrolysis of an aryl ketone. The resulting modified graphene may have electronic and/or magnetic properties.

Additional embodiments include methods to control the degree of saturation of modified graphene comprising selecting a functional group having a size suitable for forming a modified graphene having a preselected degree of saturation; and functionalizing graphene with the functional group to form the modified graphene having the preselected degree of saturation. In some aspects, the functional group modifies the magnetic properties of the modified graphene.

In some aspects, a modified graphene is the modified graphene or ferromagnetic graphene produced by the methods described herein.

Embodiments of the invention are also directed to uses. The modified graphene described herein may be used for fabrication and/or definition of a field effect transistor (FET), dielectrics, interconnects, or a ferromagnetic semiconductor. Modified graphene may also be used as a room temperature ferromagnetic semiconductor. The modified graphene may further be used to enable room-temperature anisotropic magnetoresistance in samples of the modified graphene.

### BRIEF DESCRIPTION OF THE DRAWINGS

FIG. 1 is a schematic illustration of one embodiment for modification of the electronic structure of Epitaxial Graphene (EG) which employs arylation of graphene.

FIG. 2 is a schematic illustration of one embodiment for modification of the electronic structure of Epitaxial Graphene (EG) which employs dichlorocarbene addition.

FIG. 3 is a schematic illustration of the A- and B-sub-lattices of graphene.

FIG. 4 is a schematic illustration of 1,2-functionalization on both surfaces of graphene at 1:1 or 100% coverage.

FIG. 5 is a schematic illustration of the Kekule and Clar sextets of 1,4-functionalized graphene at 2:8 or 25% coverage.

FIG. 6 is a schematic illustration of 1,4-functionalization of graphene at 2:18 or ˜11% coverage.

FIG. 7 illustrates measurements of the temperature resistance of embodiments of EG substrates before and after covalent functionalization with nitrobenzene.

FIG. 8 illustrates a baseline-corrected mid-infrared (mid-IR) spectrum of one embodiment of a nitrobenzene-functionalized EG.

FIGS. 9A-9B illustrate X-Ray Photoelectron Spectroscopy (XPS) spectra of graphene grown on the carbon face of a SiC substrate; (A) survey spectra; (b) core level spectra of C1s.

FIGS. 10A-10C illustrate XPS spectra of nitrobenzene-functionalized EG grown on the carbon-face of an SiC substrate; (5A) survey spectra; (5B) core level spectra of C1s; (5C) core level spectra of N1s.

FIG. 11 is a schematic illustration of photochemical modification of graphene with alkyl halides by generation of radicals.

FIG. 12 is a schematic illustration of one embodiment of an experimental setup for electrochemical modification of graphene.

FIG. 13 is a schematic illustration of one embodiment of oxygen functionalities in graphene introduced by electrochemical oxidation.

FIG. 14 is a schematic illustration of graphene channels formed by patterning of epitaxial graphene substrate.

FIGS. 15A-15 B illustrate the temperature dependence of the resistance of embodiments of EG substrates before and after electrochemical oxidation.

FIG. 16 is a schematic illustration of embodiments of electrochemical introduction of alkyl and aryl groups into graphene produced from pair-wise addition in the graphene A and B sub-lattices by the Kolbe reaction.

FIG. 17 is a schematic illustration of one embodiment of electrochemical introduction of alkyl groups into graphene produced from pair-wise addition in only the graphene A (or B) sub-lattice.

FIG. 18 is a schematic illustration of one embodiment of electrochemical introduction of aryl groups into graphene produced from pair-wise addition in only the graphene A (or B) sub-lattice.

FIG. 19 is a schematic illustration of one embodiment of an experimental setup for electrochemical modulation of graphene.

FIG. 20 is a schematic illustration of electrochemical modification of graphene with alkyl halides by generation of radicals.

FIG. 21 is one embodiment of a graphene-graphene oxide device.

FIG. 22 is one embodiment of a multi-channel graphene device.

FIGS. 23A-23B are atomic force microscopy (AFM) scans illustrating embodiments of epitaxial graphene samples: (A) “pristine” graphene and (B) nitrophenyl-functionalzied epitaxial graphene.

FIGS. 24A-24B illustrate Vibrating Sample Magnetometry (VSM) in-plane measurements of M-H hysteresis loops for a set of three temperature values, 2, 80, and 300 K, respectively, for a Graphene sample in (A) the original “pristine” phase and (B) “functionalized” phases. The inserts in the pristine case show a magnified view of the dependence of the magnetic moment on the magnetic field in the y-axis view (the y-scale in the inserts is from −1 to +1 μ-emu).

FIGS. 25A-25B illustrate Vibrating Sample Magnetometry (VSM) out-of-plane measurements of M-H hysteresis loops for a set of three temperature values, (a) 2, 5, and 10 K, respectively, for the pristine phase and (b) 2, 80, and 150 K, respectively, for the functionalized phase of graphene.

FIGS. 26A-26B illustrate out-of-plane magnetoresistance for a set of temperature values from 2 to 300 K for the pristine and functionalized phases, respectively.

FIGS. 27A-27B illustrate out-of-plane and in-plane magnetoresistance for a set of temperature values from 2 to 300 K for the pristine graphene.

FIG. 28 illustrates temperature dependence of in-plane and out-of-plane spin alignment.

### DETAILED DESCRIPTION OF THE PREFERRED EMBODIMENT

Recent advancements in the growth, isolation, and study of graphene suggest that graphene is a strong candidate for use in post-silicon electronics. In one example, graphene can serve as interconnects and functional logic devices in fully integrated very large scale electronic circuitry. In another example, graphene appears to hold promise for spintronic devices and circuits.

One of the major challenges to be addressed in graphene, however, is the control of electronic and magnetic properties. Such electronic properties may include, but are not limited to the necessity to engineer the band gap, conductivity, semiconductivity, carrier concentration, carrier mobility and magnetism. Reliable, reproducible and cost effective methods for this type of electronic structure and band gap engineering, large scale fabrication of electronic and magnetic devices based on graphene technology are expected to be made viable.

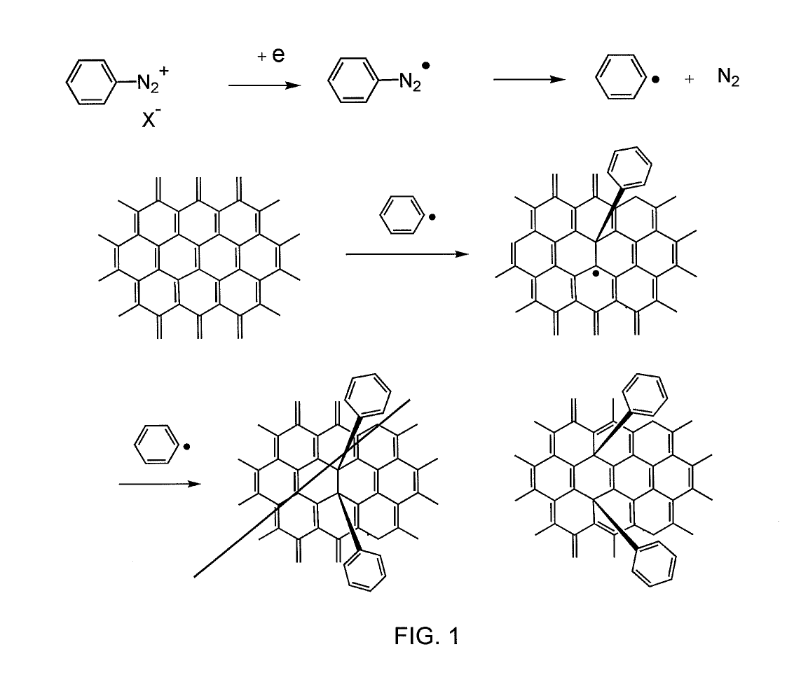

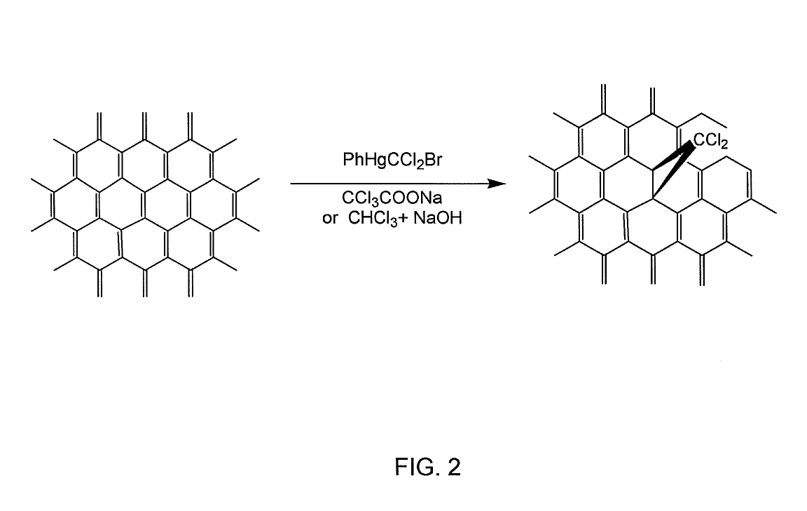

Embodiments of the present disclosure present systems and methods for chemical and electrochemical modification of the electronic and magnetic structure of graphene and particularly Epitaxial Graphene (EG). Carbon-carbon bonds are formed through the addition of organic functional groups to EG, thereby re-hybridizing the graphene atoms from sp² to sp³ (FIGS. 1 and 2 are examples). This saturation of the π-bonds of EG carbon atoms opens a local band gap in the graphene electronic structure and produces an insulating or semiconducting form of graphene; as a result it is possible to pattern a graphene wafer without physically removing the material or carrying out harsh etching reactions. We show that the local electronic structure of graphene can be controlled by the density of coverage achieved in the e.g. carbon-carbon bond formation reactions and that this can be adjusted by control of the electronic structure and bulk of the reactants. In addition we show that certain chemistries introduce electron spins into the graphene lattice, which are useful for the fabrication of ferromagnetic semiconductors. Methods for the addition of heteroatoms such as nitrogen, oxygen and fluorine and methods for substantially complete removal of the graphene carbon atoms in order to introduce insulating regions into EG are also discussed. Advantageously, the insertion of selected functionalities into EG opens the possibility of 3-D electronic architectures. These and other benefits are discussed in detail below.

The term “pristine graphene” as used herein has its ordinary meaning as known to those skilled in the art and generally includes a one-atom-thick planar sheet of sp²-bonded carbon atoms packed in a hexagonal crystal lattice. Two or more sheets of pristine graphene may also be referred to as “pristine graphene.” Types of pristine graphene which may be used to form modified graphene includes standard graphene, epitaxial graphene (EG) and chemical vapor deposition (CVD) graphene.

The term “graphene” as used herein has its ordinary meaning as known to those skilled in the art and generally includes pristine graphene and modified forms of graphene. In some aspects the modified graphene includes the modified graphene described herein or other modified forms of graphene in the art.

The term “epitaxial graphene” as used herein has its ordinary meaning as known to those skilled in the art and typically includes one layer or multilayer graphene that may be grown on a substrate, such as SiC, by e.g., vacuum graphitization. The planes in epitaxially-grown multilayer graphene are rotationally disordered and thus they are electronically decoupled and this preserves the electronic properties of an isolated graphene sheet. The term “CVD graphene” as used herein has its ordinary meaning as known to those skilled in the art and typically includes graphene synthesized via chemical vapor deposition on thin metallic layers, typically nickel layers.

The term “modified graphene” as used herein includes a modified pristine graphene including a one-atom-thick planar graphene sheet comprising sp³-bonded carbon atoms packed in a hexagonal lattice is.

In some embodiments, the sp³-bonded carbon atoms comprise a covalent carbon-carbon bond between the graphene backbone and an added group(s) such as a functional group. In additional embodiments, the sp³-bonded carbon atoms comprise a covalent carbon-heteroatom bond within the backbone of the lattice or a covalent carbon-heteroatom bond between a carbon atom in the graphene backbone and a heteroatom that is attached to the graphene backbone.

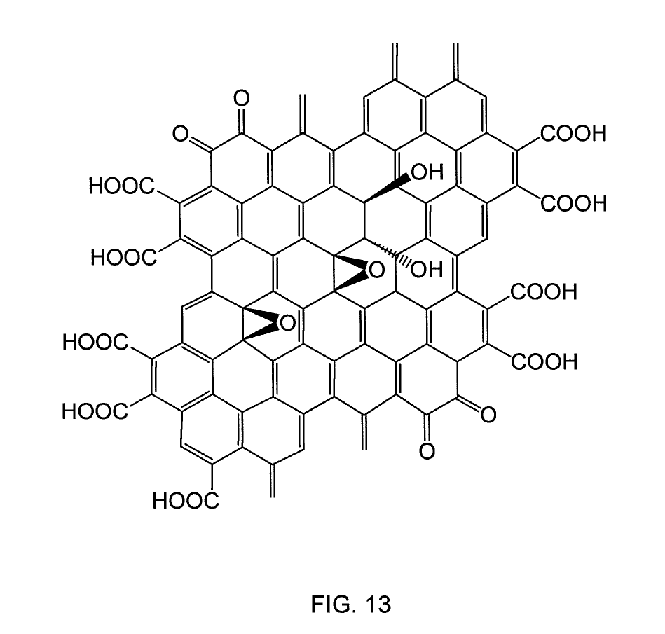

The term “functional group” as used herein has its ordinary meaning as known to those skilled in the art and typically includes an organic or inorganic group covalently attached to a carbon atom in graphene through a carbon-carbon bond, a carbon-heteroatom bond, a carbon-hydrogen bond, or a carbon-halogen bond to form the sp³-bonded carbon atom. Examples of functional groups include hydrocarbyl residues containing an optional heteroatom such as aryl or heteroaryl, an alkyl, alkenyl, or alkynyl group, or a haloalkyl, haloalkene or haloalkyne group. One functional group may form at least one carbon-carbon bond between the functional group and the graphene backbone. For example, the functional group may form two carbon-carbon bonds among two carbons within the graphene backbone and the functional group. A functional group may also form at least one carbon-heteroatom bond between a carbon in the graphene and the heteroatom of a functional group such among two carbons in the graphene backbone and a ketone as shown in FIG. 13.

The sp³-bonded carbon atom may also be incorporated into graphene by eliminating a carbon atom in the graphene backbone, and optionally adding a heteroatom, such as O, S or N, a halogen or another group at the location of the eliminated carbon atom.

The sp³-bonded carbon atom may also be incorporated into graphene by forming a carbon-heteroatom bond(s) between a carbon in the graphene and a heteroatom in an inorganic functional group such the hydroxyl group shown in FIG. 13. As used herein, “inorganic functional group” refers to a group that does not contain carbon. Examples include, but are not limited to, halo, hydroxy, ketone, NO₂ or NH₂.

In some aspects, the size of the functional group determines the surface coverage or percent of total saturation; the attachment of the first functional group to a carbon atom from the graphene lattice destabilizes, or makes more reactive, the C-neighbor and thus from the thermodynamic standpoint the next functional group should attach to the neighboring C-atom (or in ortho-position). However, a bulky group causes steric hindrance, i.e. the size of group prevents the chemical reactions that are observed in related smaller molecules, and the functional group will attach to C-atom at a para-position or even more distant C-atom. In some aspects, a large functional group results in an unpaired electron which affects the magnetic properties of the modified graphene. For example, the carbon-carbon bond in graphene is 0.142 nanometers; therefore, functional groups that are larger than the carbon-carbon bond in graphene, such as naphthalene, may be sterically hindered and thus may result in graphene having a lower saturation than a functional group that is smaller than the carbon-carbon bond in graphene. In addition, the length of the functional group may cause steric hindrance.

Saturation may also be controlled by removing functional groups from a modified graphene. For example, removal may be achieved by heating the modified graphene which may decompose some of the functional groups. Further, saturation may be controlled by adjusting the reaction time such that more functional groups may be added as the reaction time relatively increases, and fewer functional groups may be added as the reaction time relatively decreases.

The extent of saturation of the modified graphene may also affect its electronic properties. From a relative standpoint, a graphene containing a higher extent of saturation generally has a greater likelihood of containing insulating properties. For example, in one aspect, a fully saturated modified graphene will have insulating properties. In some aspects, however, insulating properties may be obtained in modified graphene having less than full saturation such as about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 50%, 59%, 60%, 61%, 62%, 63%, 64%, 65%, 66%, 67%, 68%, 69%, 70%, 71%, 72%, 73%, 74%, 75%, 76%, 77%, 70%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% saturation.

In some aspects, semiconducting properties may be obtained in modified graphene having less saturation than modified graphene that has insulating properties. In some aspects, saturation of approximately 2-40%, 5-35%, 10-30%, and particularly 11 or 28% provides semiconducting properties.

Saturation may be measured by methods known to those skilled in the art. In some aspects, saturation may be measured by using cyclic voltammetry (CV) scans. Thus, modified graphene having various functional groups can be screened based on the extent of saturation to achieve the desired properties required for a particular application.

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when so stated however, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or may contain heteroatoms within the backbone of the hydrocarbyl residue.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain one or more such as 1, 2, 3, 4, 5, 6, 7 or, 8 O, S or N heteroatoms or combinations thereof within the alkyl, alkenyl or alkynyl backbone. Haloalkyl, haloalkenyl and haloalkynyl are similarly defined but may contain one or more halogens, such as 1-10, halogens, including one or more Cl, Br, F, or I, or combinations thereof in place of hydrogens within the alkyl, alkenyl or alkynyl backbone. Examples include CX₃, CX₂(CX₂)ₙCX₃, wherein n is 1-10 and X is halo, such as X=F and n=6, or such as dichlorocarbene. These functional groups may be substituted or unsubstituted. In some aspects, the optional substituents do not interfere with the electronic or magnetic properties of the modified graphene. Typical substituents include but are not limited to inorganic substituents such as halo, hydroxy, ketone, NO₂ or NH₂.

Alkyl, alkenyl, alkynyl, heteroalkyl, heteroalkenyl, heteroalkynyl. haloalkyl, haloalkenyl and haloalkynyl groups may be substituted with similar inorganic substituents.

As used herein, the term “alkylene,” refers to a divalent hydrocarbyl group; because it is divalent, it can link two other groups together. Typically it refers to —(CH2)n— where n is 1-8 and preferably n is 1-4, though where specified, an alkylene can also be substituted by other groups, and can be of other lengths, and the open valences need not be at opposite ends of a chain. Thus —CH(Me)- and —C(Me) 2- may also be referred to as alkylenes, as can a cyclic or aryl group such as naphthalene. Alkylene includes divalent forms of alkyl, alkenyl, alkynyl, heteroalkyl, heteroalkenyl, heteroalkynyl. haloalkyl, haloalkenyl, and haloalkynyl. Examples of divalent alkylene groups include methylene, trifluoromethylene ethylene. ethenylene, ethynylene, propylene, propenylene, propynylene, and naphthylene. Where an alkylene group is substituted, the substituents include those typically present on alkyl groups as described herein.

“Aromatic” moiety or “aryl” refers to a monocyclic or fused bicyclic moiety such as phenyl or naphthyl, including those that contain one or more heteroatoms; “heteroaromatic” itself refers to monocyclic or fused bicyclic ring systems containing one or more heteroatoms selected from O, S and N. The inclusion of a heteroatom permits inclusion of 5-membered rings as well as 6-membered rings. Thus, typical aromatic systems include phenyl, naphthyl, pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroarylalkyl” refer to aromatic and heteroaromatic systems which are coupled to another residue through a carbon chain, including substituted or unsubstituted, saturated or unsaturated, carbon chains, typically of 1-6C, such as benzyl.

In order to fabricate graphene electronics such as epitaxial graphene electronics, it is advantageous to be able to controllably introduce insulating and semiconducting regions into graphene wafers. In one embodiment, insulating regions can be inserted into graphene wafers through methods such as etching away the graphene sheet, oxidation, introducing disorder into the graphene structure, and introducing bonds to atoms that are of a significantly different electronegativity than carbon, such as nitrogen, oxygen and fluorine. In another embodiment, magnetic regions and/or semiconducting regions that have well defined band structures and acceptable mobilities may be introduced by the formation of ordered arrays of carbon-carbon bonds to the graphene. EG is suitable for use in these embodiments.

Chemical and electrochemical routes for the modification of the electronic structure of graphene such as EG are discussed below. It may be understood that, while 1,2-bis addition chemistry is sometimes illustrated for structural convenience, monoadditions may be employed, with the second addition occurring at more remote sites in certain embodiments and that the pattern of additions may be controlled to encourage the formation of magnetic or nonmagnetic electronic structures in the functionalized graphene. Furthermore, while representative chemical reactions are shown, alternative organic reactions for the formation of carbon-carbon bonds may be employed to generate the illustrated structures and are within the scope of the presently disclosed embodiments.

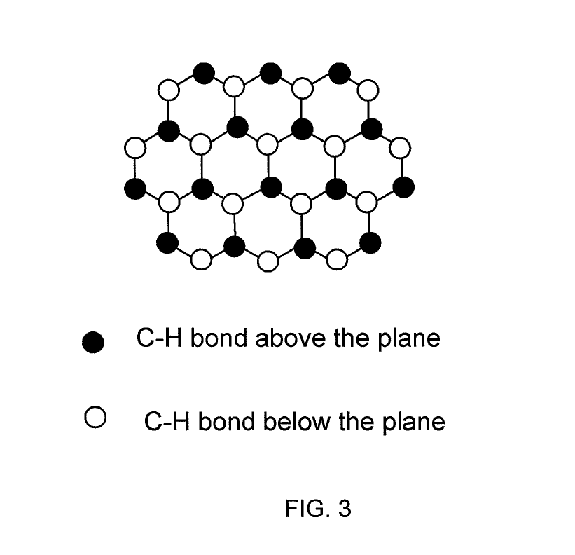

It is important to distinguish between two chemistries that have been discussed in the literature—this distinction depends on whether pair-wise reaction with two radical functionalities leads to addition of functional groups in the A sub-lattice, followed by addition in the B sub-lattice, or whether both reactions occur in the A sub-lattice (or B sub-lattice) (FIG. 3). Theoretically, pair-wise radical addition reactions which occur in both the A and B sub-lattice lead to semiconducting structures without free spins [Boukhvalov, D. W.; Katsnelson, M. I., Tuning the Gap in Bilayer Graphene using Chemical Functionalization: Density Functional Calculations. Phys Rev. B 2008, 78, 085413]. Whereas pair-wise radical reactions that both occur in either the A sub-lattice (or the B sub-lattice), lead to free spins that can couple ferromagnetically [Yazyev, O. V.; Helm, L., Defect-Induced Magnetism in Graphene. Phys Rev. B 2007, 75, 125408. The coupling between ferromagnetic regions may be antiferromagnetic, offering a wide range of available magnetic states and the possibility of ferromagnetic semiconductors. The theoretical studies do not show how to make graphene with sp³ bonds that provide the desired electronic and mechanical properties as described herein. Difficulty in making additions to graphene arises from the relatively inert nature of the carbon-carbon bonds in graphene. In addition, electronic or magnetic properties of functional group additions could not be predicted based on the addition of functional groups to a non-graphene material.

For example, graphite is a three-dimensional material and carbon nanotubes are one dimensional material. Carbon nanotubes are more reactive than graphene due to the local strain induced by the curvature-induced pyramidalization and misalignment of the π-orbitals of the carbon atoms. In graphite, only the top layer can be functionalized and this has negligible effect on its properties. especially the transport properties, whereas the functionalization of graphene strongly affects the electronic structure and electronic properties of the material.

Thus, modifications of graphite or carbon nanotubes cannot predict the feasibility or outcome of the same modifications in graphene. In addition, modifications to graphite or carbon nanotubes do not provide the electronic or mechanical properties desired in graphene having the same modifications. In some aspects, the described functionalization approach modifies the electronic properties of graphene from semimetallic to semiconducting and isolating based on the degree of functionalization or surface coverage.

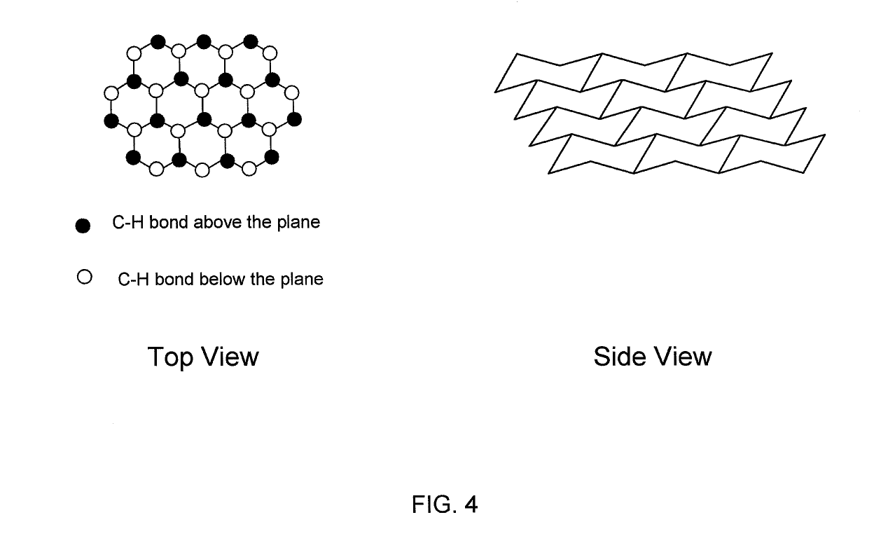

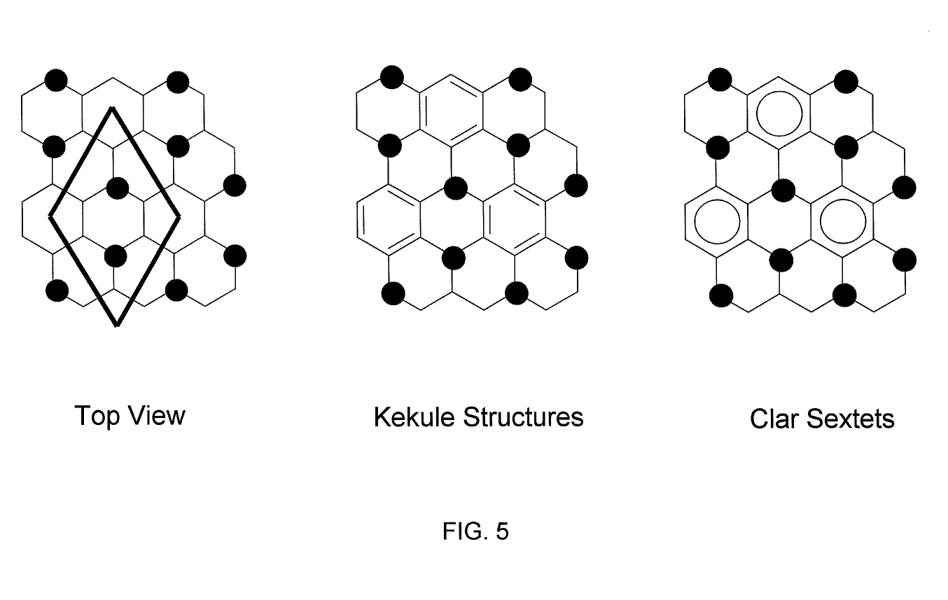

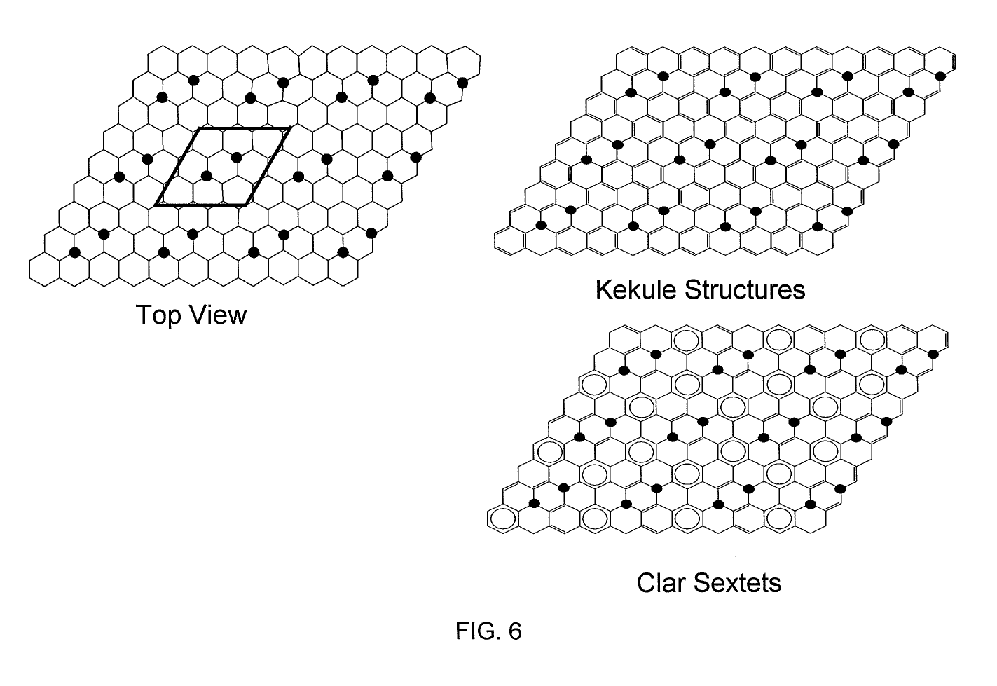

FIGS. 4 to 6 illustrate the use of the degree of coverage to control the level of unsaturation in the graphene electronic structure. Based on the standard concepts of organic chemistry and solid state physics, it is to be expected that those graphene structures with more unsaturation and with well defined conjugated pathways through the graphene lattice (FIG. 6) will be associated with a lower band gap and higher mobilities than the structures which are completely saturated (FIG. 4), or which are partially unsaturated but have not retained fully conjugated pathways through the lattice (FIG. 5). In the case of the 2:8 coverage (FIG. 5), it is clear that the final structure consists of isolated benzene rings in which the conjugation is interrupted by saturated carbon atoms at every position and this is reflected in band structure calculation, which shows a band gap of ˜2 eV. [Boukhvalov, D. W.; Katsnelson, M. I., Tuning the Gap in Bilayer Graphene using Chemical Functionalization: Density Functional Calculations. Phys Rev. B 2008, 78, 085413] On the other hand the 2:18 coverage (FIG. 6), leaves a conjugated network of pyrene molecules, which are joined at the 1, 3, 6, 8-positions. Thus it is possible to use chemistry to generate a range of structures that differ only in the density of coverage which encompass fully saturated (FIG. 4), partially saturated (FIG. 5), and conjugated (FIG. 6) structures, which will offer distinct electronic properties that will be reflected in their transport properties. Clearly many other coverages are possible leading to a large variety of locally patterned electronic structures and devices and thus the present FIGS. 4-6 are illustrations of a large family of useful structures.

The term “well defined conjugated pathways” as used herein has its ordinary meaning as known to those skilled in the art and typically refers to pathways within the modified graphene wherein electrons flow and thus form a modified graphene with a relatively low band gap and high mobility.

The term “ill defined conjugated pathways” as used herein has its ordinary meaning as known to those skilled in the art and typically refers to modified graphene having localized electrons and thus form modified graphene with a relatively larger band gap and lower mobility.

The term “continuous conjugation of sp² carbon atoms” as used herein has its ordinary meaning as known to those skilled in the art and typically refers to pathways within the modified graphene wherein electrons flow and thus form a modified graphene with a relatively low band gap and high conductivity.

The term “absence of continuous conjugation of sp² carbon atoms” as used herein has its ordinary meaning as known to those skilled in the art and typically refers to modified graphene having localized electrons and thus form modified graphene with a relatively larger band gap and lower conductivity.

Graphene itself provides the best example of continuous conjugation in that every conjugated sp² hybridized carbon atom is bonded to 3 other hybridized sp² carbon atoms.

FIG. 6 shows a structure that still maintains continuous conjugation of sp² carbon atoms—that is, there is an unbroken pathway of such carbons through the structure.

Graphene (FIGS. 3 and 4) are the extreme opposite as there are no conjugated sp² carbon atoms remaining in the structure.

FIG. 5 shows a structure in which there is an absence of continuous conjugation of sp² carbon atoms—that is, there is no continuous pathway of such carbons through the structure.

Below we show a range of reactions, which may be used for accomplishing these chemical modifications, most of which depend on the generation of radicals that are sufficiently reactive to add to the graphene basal plane carbon atoms. Two of these reactions are particularly suited to electrochemistry (although under certain circumstances they can occur by spontaneous electron transfer): the reduction of diazonium salts and the oxidation of carboxlyates (Kolbe reaction). For these cases we illustrate a number of possible reactants and they offer particular opportunities.

### EXAMPLES

### Example 1

### Dichlorocarbene Addition and Arylation

FIGS. 1 and 2 illustrate embodiments of methods for modification of the electronic structure of EG using dichlorocarbene addition and arylation.

### Dichlorocarbene Addition to Epitaxial Graphene

In one embodiment of the dichlorocarbene addition method, a substrate of epitaxial graphene is substantially immersed in a first solution comprising about 0.001 mg/mL to 10 mg/mL triethyl benzylammonium chloride (phase transfer catalyst, PTC) in chloroform or a mixture of chloroform and water at a ratio of about 1:1 to 2:1. The reaction vessel is purged with an inert gas, for example, argon. A second solution of about 1 g to 50 g NaOH in about 10 mL to 50 mL chloroform and about 1 mL to 50 mL water is added slowly to the reaction vessel. The resulting mixture is refluxed at a temperature of about 53° C. for between about 1 hour and 36 hours and subsequently cooled to about room temperature. The substrate is then removed from the solution and washed with chloroform and water.

In one embodiment, approximately 100 mg of triethyl benzylammonium chloride and about 30 ml of chloroform are employed to prepare the first solution. The first solution may be further placed in a 500 mL three-neck round-bottom flask equipped with a condenser. The flask is purged with argon for 30 min, and about 20 g NaOH, 30 ml of chloroform, and about 10 ml of deionized water are employed to prepare the second solution. The mixture is then refluxed at a temperature of about 53° C. for about 24 hours. The reaction vessel is subsequently cooled to about room temperature and the substrate is removed from the solution and washed with chloroform and water.

In another embodiment of the dichlorocarbene addition method, PhHgCCl2Br is employed. In one embodiment, about 10 mg to 1 g PhHgCCl2Br is dissolved in about 10 mL to 100 mL dichlorocarbene or toluene. The epitaxial graphene substrate is substantially immersed in the solution and heated at about 85 to 95° C. under an inert gas for about 0.1 to 48 hours. The solution is allowed to cool to room temperature, after which point the substrate is removed and washed with acetone. In one embodiment, about 1.7 g of PhHgCCl—2Br is dissolved in about 75 mL dichlorobenzene and the solution is heated to about 85° C. under argon for 48 hours. The solution is cooled to room temperature, the substrate is removed and washed with acetone.

### Spontaneous Grafting of Aryl Groups to Epitaxial Graphene:

An embodiment of a method for the modification of the electronic structure of EG by spontaneous grafting of aryl groups is also illustrated in FIG. 1. In one embodiment, grafting reactions may be carried out in a solution of a diazonium salt in acetonitrile (ACN) at about room temperature in substantial absence of light and air. Examples of such diazonium salts may include, but are not limited to, 4-nitrobenzenediazonium (NBD) tetrafluoroborate, 4-carboxybenzene diazonium tetrafluoroborate, and 4-bromobenzenediazonium tetrafluoroborate. The graphene substrate is substantially immersed in an ACN solution of about 1 to 100 mM diazonium salt and about 0.01 to 0.2 M electrolyte for times ranging between about 1 second to about 24 hours. Examples of the electrolyte may include, but are not limited to, tetrabutylammonium hexafluorophosphate and tetrabutylammonium tetrafluoroborate. The substrate is subsequently removed from the solution and washed with ACN and acetone. In one more specific example, the epitaxial graphene grown on the carbon (C)-face of SiC was immersed in an approximately 10 mM solution of nitrobenzenediazonium tetrafluoroborate in about 5 mL ACN with about 0.1 M of tetrabutylammonium hexafluorophosphate for about 1.5 hours in substantial absence of light and air. Subsequently, the substrate was rinsed with ACN and acetone to form epitaxial graphene modified with nitrophenyl.

In alternative embodiments, the reaction may also be carried out in about 0.001 to 1.0 M aqueous sulfuric acid (H₂SO₄), employing the chloride salt of the diazonium cation. In one embodiment, approximately 0.14 M H₂SO₄ may be so employed.

The successful functionalization of the graphene may be examined through measurements which may include, but are not limited to: cyclic voltammetry, impedance spectroscopy, transport measurements, mid-infrared spectroscopy (mid-IR), and X-Ray Photoelectron Spectroscopy (XPS).

In one embodiment, the temperature dependence of resistance was measured using a custom-made, variable temperature probe. The probe was cooled using liquid helium and the temperature was measured using a Lake Shore 340 temperature controller. The resistance was measured by the four-point method using a Keithley 236 source-measure unit and two Keithley 6517 electrometers controlled by custom Labview Software.

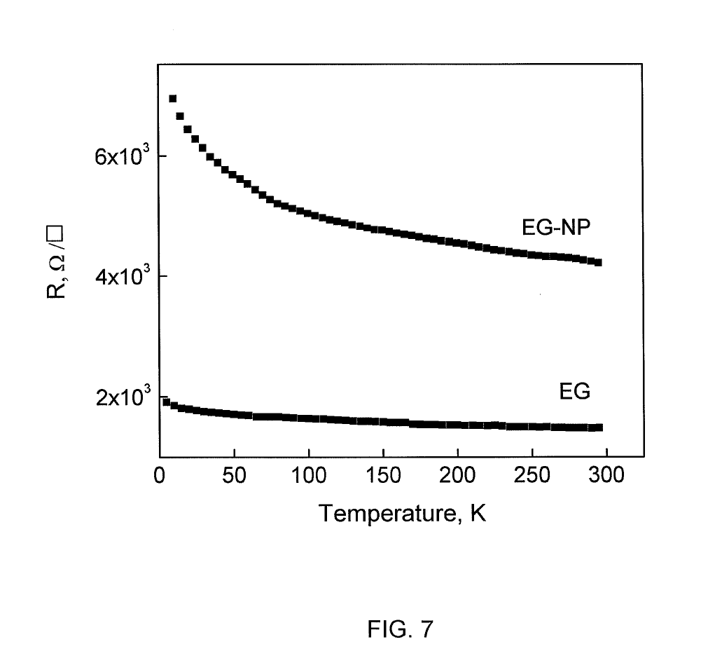

FIG. 7 illustrates the measured temperature dependence of resistance for embodiment of one substrate which was subjected to the nitrobenzene grafting treatment. These measurements show that grafting of nitrobenzene to the surface of epitaxial graphene increases the temperature dependence of resistance.

Comparison of the room temperature resistance of the pristine and nitrophenyl-functionalized EG samples showed an increase of the resistance of EG from about 1.5 kΩ/□ before the functionalization to 4.2 kΩ/□ after the attachment of nitrophenyl groups. In addition the very weak temperature dependence of the resistance of pristine EG is substantially increased on NP-functionalization. The increase in the measured resistance at about room temperature and the increased temperature coefficient of resistivity on cooling are both consistent with an increase in the EG band gap and show that the functionalization can create semiconducting regions in the graphene layer.

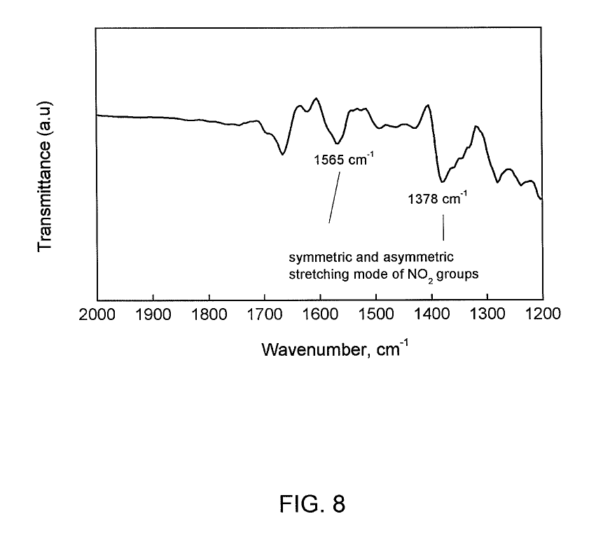

The formation of covalent bonds between the C atoms from the graphene surface and nitrobenzene may be detected by mid-IR spectroscopy of the nitrobenzene-functionalized epitaxial graphene, as illustrated in the spectrum given in FIG. 8. The transmittance spectrum was taken using a Nicolet Nexus 670 FT-IR spectrometer at a resolution of about 8 cm⁻¹, and the spectrometer chamber was purged with nitrogen during the measurements. The bands at about 1565 cm⁻¹ and 1378 cm⁻¹ are assigned to the anti-symmetric and symmetric N—O stretching vibrations of the nitro group in nitrobenzene.

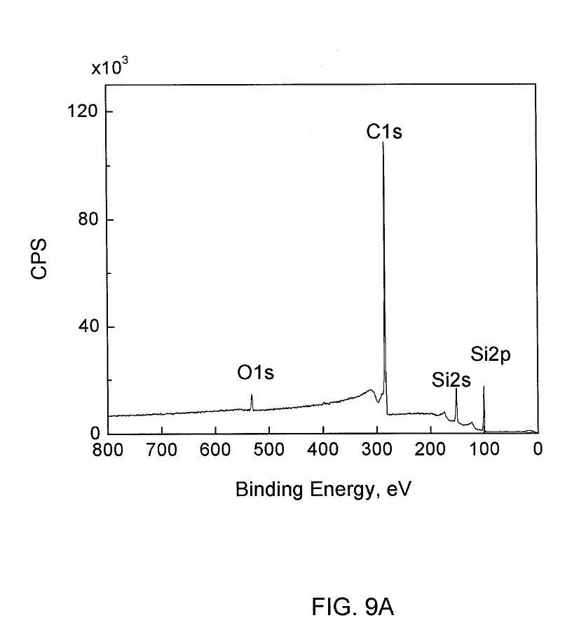

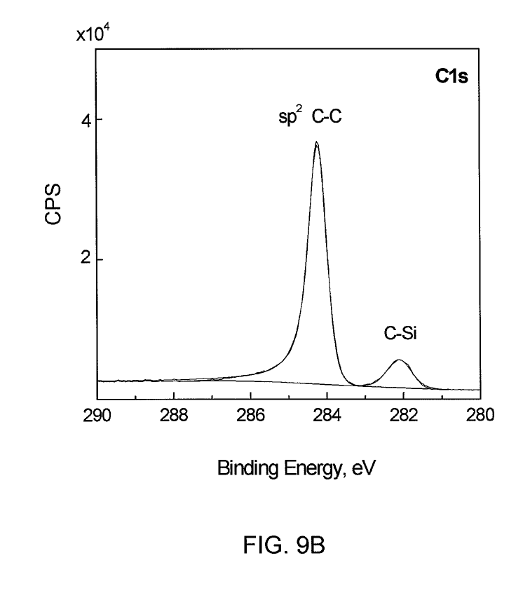

The covalent attachment of the nitro groups to the EG surface may be further illustrated by XPS measurements of graphene and nitrobenzene-functionalized graphene. FIG. 9A illustrates a survey spectrum of one embodiment of pristine EG grown on the carbon face of a SiC substrate, while FIG. 9B illustrates a core level C1s spectrum. As shown in FIG. 9A, the as-grown graphene demonstrates mainly the presence of carbon (C), trace amounts of oxygen (O) and silicon (Si) from the SiC substrate. The C1s spectrum of FIG. 9B shows a strong peak at about 284.24 eV which may be attributed to the binding energy (BE) of sp² hybridized C atoms.

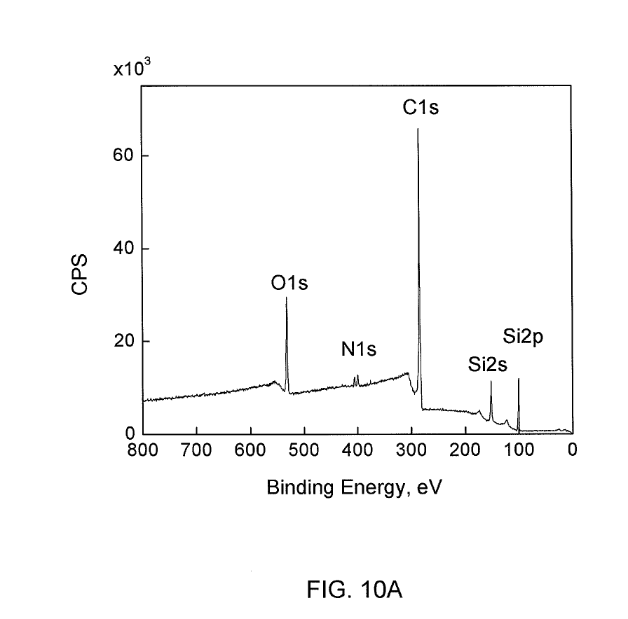

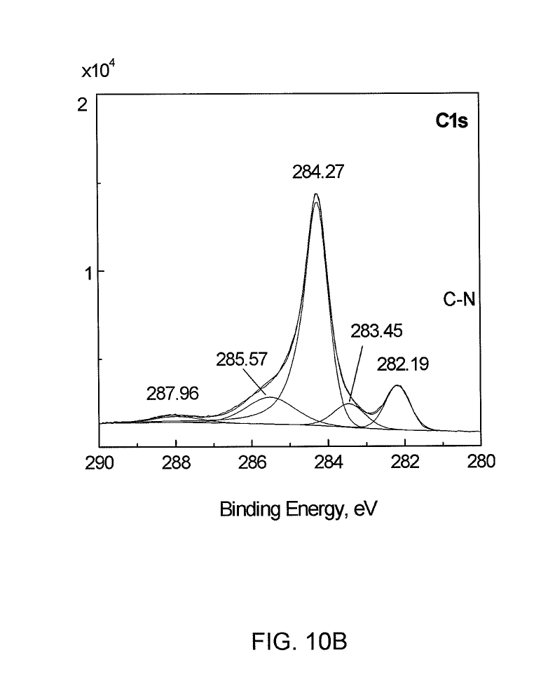

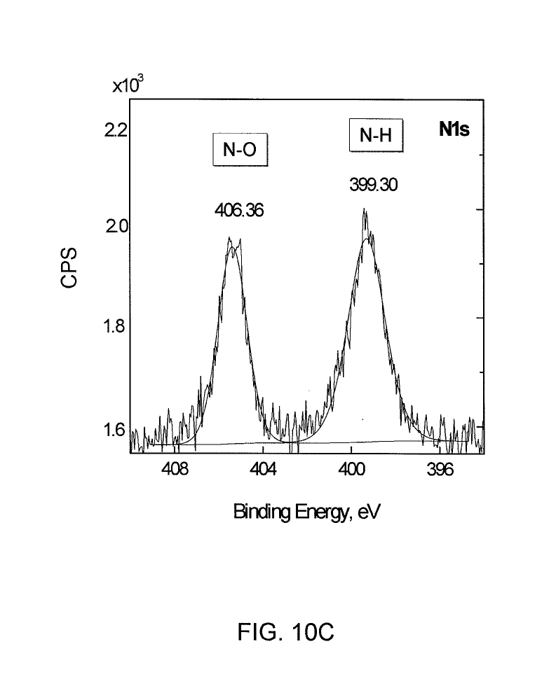

XPS spectra of nitrobenzene-functionalized graphene are presented in FIGS. 10A-10C and clearly illustrate the presence of nitrogen. The N1s spectra (FIG. 10C) shows two peaks at about 399.30 eV and 405.36 eV, which are attributed to the presence of the nitro group. Notably, the C1s peak (FIG. 10B) is significantly broadened as compared to the C1s peak of the as-grown graphene (FIG. 9B) and may be deconvoluted into several components: a peak at about 283.45 eV due to C—H bonds and a peak at about 287.96 eV due to C—N bonds, together with peaks due to various C—C bonds. These results confirm the successful spontaneous grafting of nitrobenzene groups to the epitaxial graphene surface.

### Arylation of Epitaxial Graphene (In-Situ Grafting):

In a further embodiment, graphene may be functionalized with in-situ generated diazonium cations. In one embodiment, a solution of about 0.1 g to 10 g of a metallic nitrite, for example sodium nitrite, in water (1 to 100 mL), is added to a solution of about 0.1 g to 20 g arylamine (for example p-nitroaniline) in aqueous mineral acid. Examples of such mineral acids may include, but are not limited to, hydrogen chloride and sulfuric acid. In another embodiment, a mixture of about 0.1 to 10 g arylamine (for example, p-nitroaniline) and about 0.1 to 10 g of a nitrite, such as sodium nitrite, in an aqueous medium is added to an excess of mineral acid, such as concentrated hydrogen chloride or sulfuric acid. The reactions are carried out for 1 second to 2 hours. In either case, the reaction is carried out in the presence of a graphene substrate. After the reaction, the substrate is washed with water and acetone.

### Example 2

### Radical Addition and Graft Polymerization

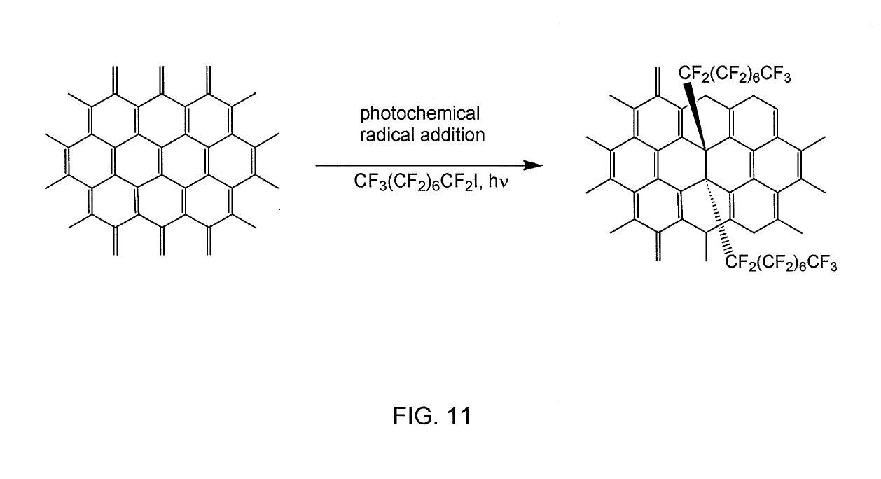

FIG. 11 illustrates further embodiments of methods for modification of the electronic structure of graphene: comprising radical addition and graft polymerization of graphene.

### Radical Addition:

In one embodiment of a radical addition process for chemical modulation of the electronic properties of epitaxial graphene, a substrate supporting epitaxial graphene is substantially immersed in a approximately 10- to 200-fold excess of heptadecafluorooctyl iodine (CF₃(CF₂)CF₂I) dissolved in about 10 to 100 mL 1,1,2,2-tetrachloroethane (TCE) and is illuminated with a medium pressure mercury lamp of about 50 to 350 W for about 1 second to about 8 hours. In one embodiment, an approximately 200-fold excess of heptadecafluorooctyl iodine dissolved in about 10 mL TCE is employed. In a further embodiment, the illumination source may comprise an approximately 150 W mercury lamp. Following the illumination, the substrate is removed from the solution and washed with TCE and acetone.

### Graft Polymerization:

In an embodiment of a graft polymerization process for chemical modulation of the electronic properties of epitaxial graphene, about 10 mg to 1 g paracyclophane is sublimed at about 190 to 210° C. under vacuum into a furnace, such as a tube furnace, which is set to a temperature where the xylylene co-monomer is formed, for example, about 600 to 800° C. In one embodiment, the furnace is operated at a temperature of about 650° C. The xylylene vapor is passed into a reaction vessel containing a substrate with EG, which is cooled to about room temperature to −78° C. Subsequently, the vessel is allowed to warm to room temperature and the substrate is removed.

### Experimental Set-Up for Small Area Electrochemistry of Epitaxial Graphene:

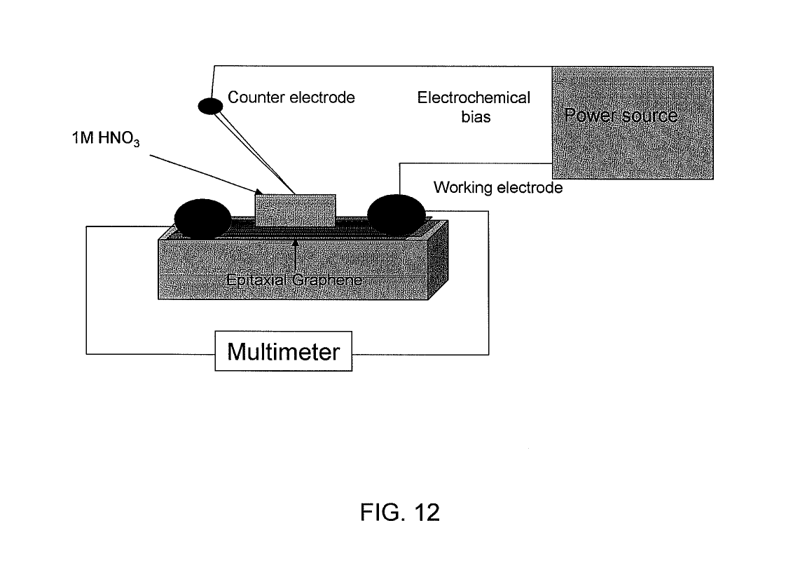

FIG. 12 represents a schematic illustration of one embodiment of an experimental setup for the electrochemical modification of graphene. The device comprises a working electrode, a reference electrode, and a counter electrode in electrical communication with a potentiostat. In one embodiment, epitaxial graphene was used as the working electrode in an electrochemical cell containing an acid. In one embodiment, the acid comprises about 1M nitric acid (HNO₃). The EG was contacted with a conductive paint, such a silver paint in order to attach electrical leads to the EG. The silver paint contacts are further sealed with epoxy or other chemically inert material. The EG electrode was biased against the reference electrode in order to oxidize the graphene and to introduce oxygen functionality as graphene oxide, as illustrated in FIG. 13. These oxygen functional groups introduce sp³ character into the graphene sheet, opening a band gap.

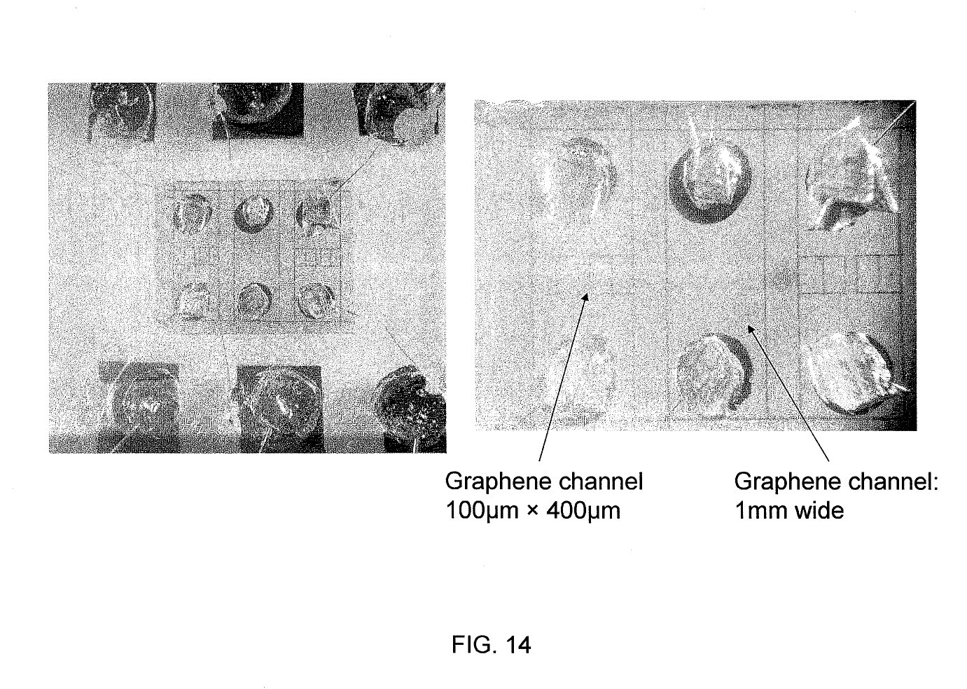

In additional embodiments, a channel as shown in FIG. 14 may be introduced beforehand by techniques such as focused ion beam milling, electron beam (e-beam) lithography or selectively masking of the EG layer so as to control the access of the reagent to the graphene carbon atoms. Beneficially, it is possible to vary the band gap by adjusting the width of the channel which is oxidized.

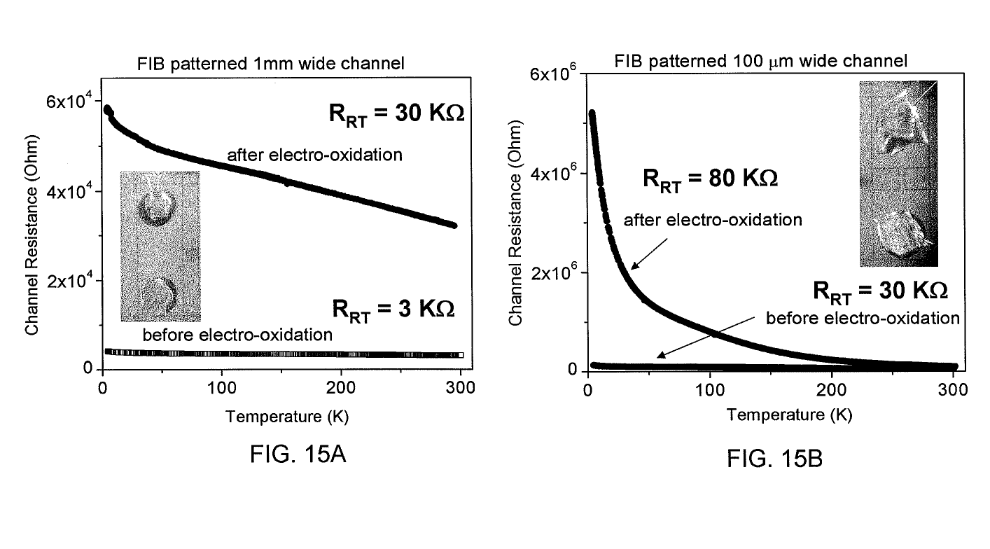

FIG. 15 shows that the temperature dependence of resistance of electrochemically oxidized graphene substrates significantly increased as compared to pristine graphene.

It should be noted, however, that more aggressive oxidation may lead to substantially complete removal of the graphene layer and the introduction of insulating regions.

As discussed below, similar electrochemical functionalization may be employed to introduce carbon-carbon bonds via radical chemistry on the graphene surface.

### Example 3

### Electrochemical Attachment of Alkyl and Aryl groups

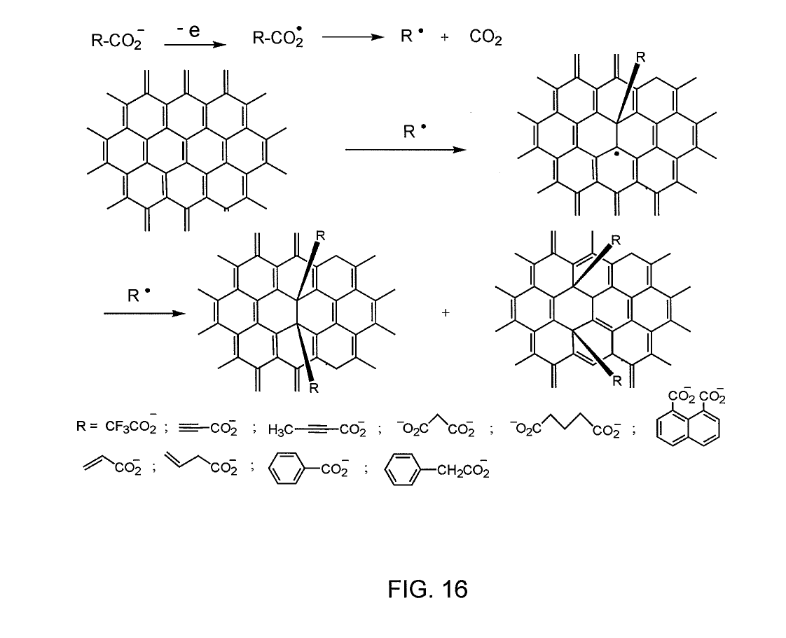

FIG. 16 illustrates embodiments of methods for modification of the electronic structure of EG by attachment of alkyl and aryl groups by the Kolbe Reaction. In one embodiment, the reaction may be carried out using cyclic voltammetric (CV) scans to a potential which is slightly positive with respect to the cyclic voltammetric peak. In an alternative embodiment, the reaction may be carried out by controlled potential electrolysis at positive potentials.

### CV Scans:

In one embodiment, the derivatization may be carried out in an approximately 1-5 mM solution of a carboxylate (generated from the carboxylic acid or anhydride by addition of an equivalent of tetrabuylammonium hydroxide) in acetonitrile containing about 0.01-0.1 M of a supporting electrolyte. Examples of such electrolytes may include, but are not limited to, n-Bu₄NBF₄ and Bu₄NPF₆. The graphene is immersed in the solution and functions as a working electrode. In further embodiments a metal, such as platinum or gold, may be used as a counter electrode. In further embodiments, a saturated calomel electrode (SCE) or Ag/AgCl may be employed as a reference electrode. A potential which is positive with respect to the cyclic voltammetric peak of the carboxylate is applied to the working electrode which is placed adjacent to the graphene and a current density of about 0.25 A/cm² or higher is provided.

### Electrolysis:

In an embodiment of an electrolysis method for chemical modulation of graphene, about 0.001 to 1M carboxylic acid and about 0.001 to 2 mol % potassium hydroxide are dissolved in methanol in order to generate the conjugate base of the carboxylic acid. In one embodiment, approximately 0.1 M carboxylic acid and about 3.5 mol % potassium hydroxide are employed. The carboxylic acids selected to generate conjugate bases may include, but are not limited to, trifluoracetic acid, propiolic acid, 2-butynoic acid, malonic acid, succinic acid, glutaric acid, naphthalic anhydride, acrylic acid, crotonic acid, benzoic acid, phenyl acetic acid (see Kolbe reaction, FIGS. 16, 17 and 18). The graphene is immersed in the solution and connected to a power source so as to serve as an anode. A cathode, comprising materials such as platinum, steel, or nickel, is placed adjacent to the graphene and a current density of greater than about 0.25 A/cm² is provided. A cooling bath is employed to maintain the temperature of the solution between about 10 to 45° C. The electrolysis is run for a period of 1 second to 10 hours. Subsequently, the substrate is rinsed with methanol.

### Example 4

### Electrochemical Attachment of Aryl Groups

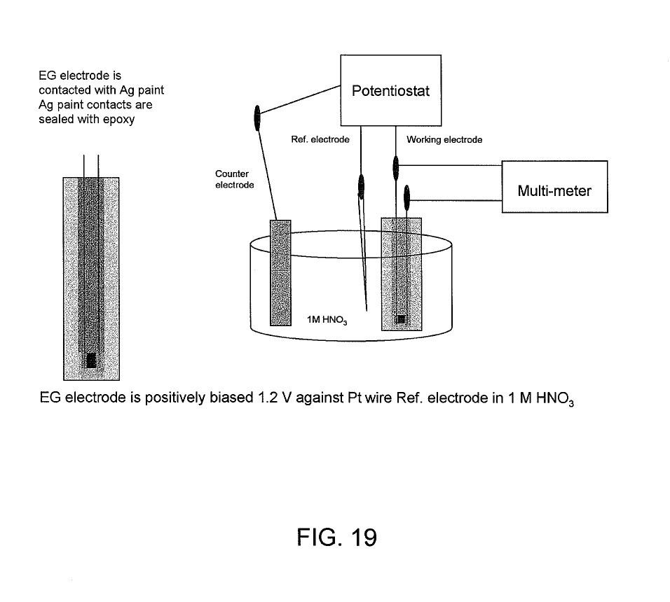

FIG. 19 illustrates further embodiments of methods for modification of the electronic structure of EG by electrochemical attachment of aryl groups through CV scans and electrolysis.

### CV Scans Using Diazonium Salts:

In one embodiment of this process, a graphene substrate is used as a working electrode. The reaction is carried out in a three-electrode cell, with a reference electrode comprising Ag/AgCl and a counter electrode comprising platinum wire. A substrate with the EG is immersed in an acetonitrile solution of a diazonium salt in the presence of a supporting electrolyte. In one embodiment, the diazonium salt comprised p-nitrobenzenediazonium tetrafluoroborate. In additional embodiments, the electrolyte may include, but is not limited to, tetra-n-butlyammonium tetrafluoroborate. In further embodiments, about 1 to 100 mL acetonitrile, about 0.001 to 1M diazonium salt, and about 0.001 to 1 M electrolyte may be employed. Aryl groups may be attached to the EG by the reduction of the diazonium salt in an inert environment, such as nitrogen, by scanning the potential between about +1.0 and −1.0 V (vs. Ag/AgCl) at a scan rate between about 1 to 1000 mV/s. In one embodiment, the scan rate is about 200 mV/s. After the modifications, the substrate may be rinsed with acetonitrile and acetone.

### Electrolysis Using Diazonium Salts:

In a further embodiment, aryl groups may be grafted to the graphene surface by electrolysis using a potential, which is more negative than the reduction potential of a selected diazonium salt. A negative potential is applied to the graphene, which functions as a cathode, while a platinum wire may be employed as an anode. In one embodiment, the potential ranges between about 0V to −1.5V. The electrochemical reaction of the diazonium salt may be conducted in an aprotic solvent, such as acetonitrile, dimethylformamide, dimethylsulphoxide or benzonitrile, or a protic solvent in an acid medium having a pH less than about 2 including, but not limited to, sulfuric, hydrochloric, nitric, nitrous, phosphoric, and tetrafluoroboric acids. The concentration of the diazonium salts is between about 0.001 and 1 M. In further embodiments, the electrolysis may take place between about 1 s to 2 h.

### Example 5

### Electrolytic Coupling of Alkyl and Aryl Halides with Graphene:

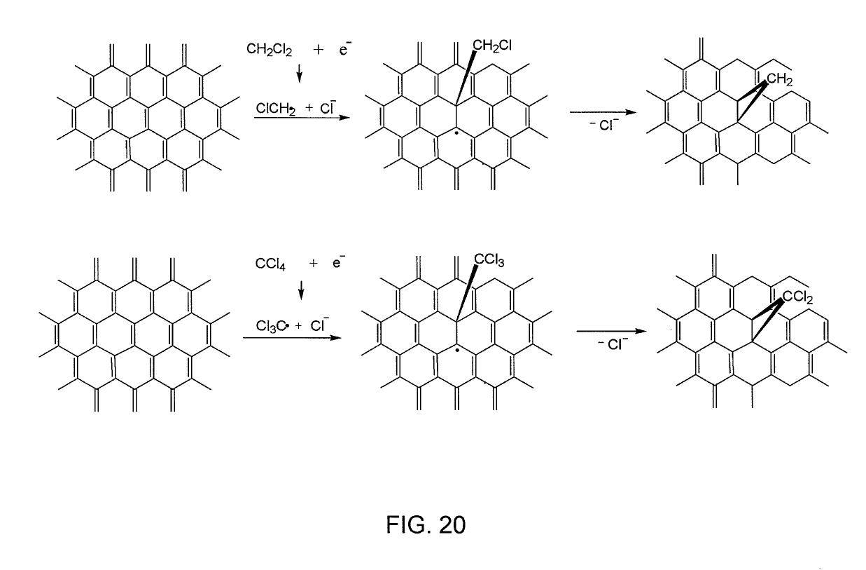

FIG. 20 illustrates further embodiments of methods for modification of the electronic structure of EG by electrolytic coupling of alkyl halides. An alkyl halide is dissolved in about 5 to 50% aqueous dimethylformamide or acetonitrile to obtain a solution having a concentration ranging between about 0.001 and 1 M. The supporting electrolyte is a tetraalkylammonium salt, which may include, but is not limited to, tetraethylammonium perchlorate, tetrabutylammonium bromide, and tetrapropylammonium fluoroborate. The concentration of the tetraalkylammonium salt may range between about 0.001 to 1 M. The graphene, which may serve as a cathode, is substantially immersed in the solution and the solution is purged with an inert gas, such as nitrogen or argon, for about 5 min to 2 h. The reaction is configured to take place at a potential that is more negative than the first reduction potential of the alkyl halide. In one embodiment, the potential ranges between about −1 V to −5V. The reaction may proceed for about 1 s to 24 h.

### Example 6

### Electrolytic Coupling of Aryl Ketones with Graphene:

The electronic structure of EG is modified by electrolytic coupling with the reduced form of aryl ketones. An aryl ketone is dissolved in about 5 to 50% aqueous dimethylformamide or acetonitrile to obtain a solution having a concentration ranging between about 0.001 and 1 M. The supporting electrolyte is a tetraalkylammonium salt, which may include, but is not limited to, tetraethylammonium perchlorate, tetrabutylammonium bromide, and tetrapropylammonium fluoroborate. The concentration of the tetraalkylammonium salt may range between about 0.001 to 1 M. The graphene, which may serve as a cathode, is substantially immersed in the solution and the solution is purged with an inert gas, such as nitrogen, for about 5 min to 2 h. The reaction is configured to take place at a potential that is more negative than the first reduction potential of the aryl ketone. In one embodiment, the potential ranges between about 0V to −5V. The reaction may proceed for about 1 s to 24 h. In one embodiment 0.1 g of benzophenone is reduced in 50 mL acetonitrile containing 1 mL of water at −2V for a length of time necessary to obtain the desired EG surface coverage with the 1-hydroxy-1-phenyl-benzyl group.

### Graphene Devices:

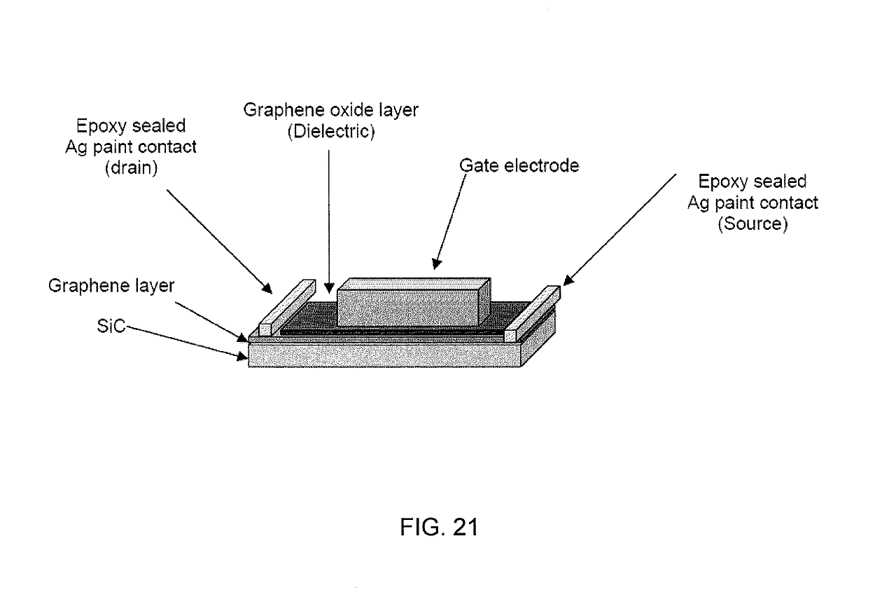

FIG. 21 illustrates one embodiment of a graphene device. The device comprises a silicon carbide substrate which supports a graphene layer, a source, a drain, a gate, and a dielectric. In certain embodiments, the dielectric may comprise a graphene oxide layer.

Using this device, the FET properties of the functionalized EG are examined. In one embodiment, at certain values of the source-drain voltage, the current through the device (source to drain), is measured as a function of the gate voltage and the results interpreted to obtain the threshold voltage, transconductance and mobility of the carriers in the device. The FET properties may also be examined as a function of gate voltage.

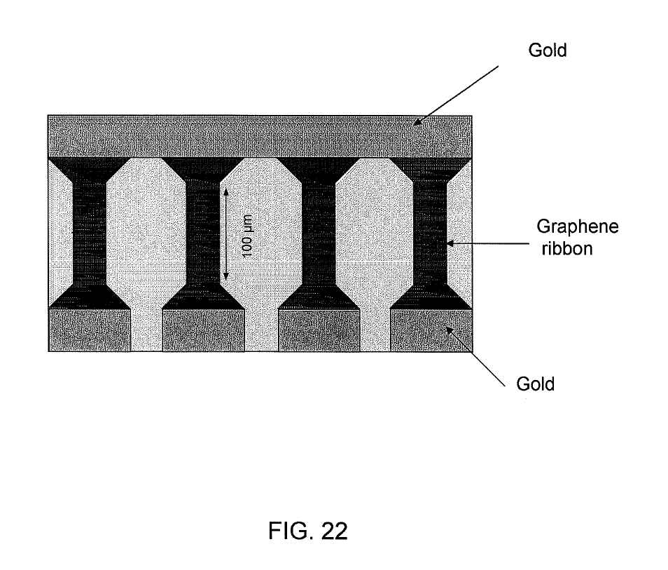

FIG. 22 illustrates one embodiment of a multi-channel graphene device. The device comprises ribbons of functionalized graphene which are in electrical communication with gold contacts. In one embodiment, the graphene ribbon has a length of about 0.01 to 1000 μm and a width of about 0.01 to 100 μm. In further embodiments, the length and width of the graphene ribbon are about 100 μm and 1 μm, respectively.

In a further approach, the graphene sheet may be patterned by covalently grafting organic functional groups via electrochemically initiated or spontaneous reduction of aryldiazonium salts. In one embodiment, nitrophenyl and aminophenyl groups may be attached to EG. In further embodiments, longer chain organic molecules may be attached in a similar fashion. Based on the attached functional group, the graphene properties may be modulated in order to obtain semiconducting channels for various bandgaps as well as an insulating surface that may serve as a gate in a FET device or as a sensor for particular chemical analytes. A variety of patterns can be readily obtained using an elastomeric stamp, such as poly(dimethylsiloxane) (PDMS) that is inked in a solution of aryldiazonium salt.

This approach provides a variety of advantages. In one aspect, the process is relatively simple. In further embodiments, the process may be carried out in aqueous and organic solutions of diazonium salts. In additional embodiments, the degree of surface coverage may be controlled. In other embodiments, a variety of patterns may be printed. In further embodiments, standard lithographic techniques may be employed to pattern large surfaces, such as wafers. In additional embodiments, channels and semiconducting and insulating regions may be introduced by controlled chemical functionalization.

### Functionalized Graphene as Room-Temperature Magnetic Semiconductor (MS):

One of the most significant and practical advantages of chemically functionalized graphene is its property to act as a room-temperature magnetic semiconductor (MS). Moreover, through chemical functionalization, it is possible to tailor MS temperatures to match specific features sought in various next-generation electronics, magneto-electronics and spintronics applications.

In some aspects, spintronic devices require the ability to impose a magnetic polarization on the electric current and in turn to control magnetic fields by an electric current and to simultaneously have semiconductor properties (necessary for controlling the charge-based electric currents). Thus, in one aspect, it is necessary to achieve ferromagnetism in a semiconductor at room temperature to enable the fabrication of many different classes of spintronic devices.

In one embodiment, functionalized graphene shows all of the desired propertiers: (I) magnetic at room temperature (as measured through MH magnetometry); (ii) semiconductor (transport measurements). The ability to control electric properties through magnetism may be also shown through the presence of non-zero magnetoresistance.

It is a special time for the semiconductor electronics industry. For the first time, scaling cannot be used to further advance core technologies. With the transistor gate thickness being scaled to the sub-10-nm range, the exponentially increased charge leakage because of quantum-mechanical tunneling makes semiconductor devices inadequate. On the contrary, magneto-electronics and spintronics devices that exploit not only the electric charge but also the magnetic spin of the electron have the potential to be scaled to sub-1-nm range. Moreover, spintronics promises to give birth to a new breed of computing devices with such important advantages as non-volatility, data densities above 100 terabit/in2, data rates in the terahertz range, and negligibly small power consumption. However, to enable many potential breakthrough applications, new materials that combine both magnetic and semiconducting properties at room temperature need to be discovered. Since early 1990 s, extensive research has been conducted to explore the feasibility of using (III, Mn) V diluted magnetic semiconductors (DMS) as the candidates for room-temperature ferromagnetic semiconductors. Unfortunately, increasing Curie temperatures from the current record of approximately 200 K remains the main open question with DMS materials.

Recent theoretical and experimental efforts have indicated that graphene, defined as a few single layers of graphite, may indeed be a candidate for the long-sought-after material. Theoretical calculations indicated that a “defective” graphene sheet was likely to be simultaneously semiconducting and magnetic or, in other words, act as a magnetic semiconductor (MS) [L. Pisani, B. Montanari, N. M. Harrison, “A defective graphene phase in predicted to be a room temperature ferromagnetic semiconductor,” New Journal of Physics 10, 033002 (2008); O. V. Yazyev, L. Helm, “Defect-induced magnetism in graphene,” Phys. Rev. B 75, 125408 (2007)]. Recently, weak ferromagnetism at room temperature was observed in graphene samples via M-H measurements. However, the following open questions remain: (i) Is ferromagnetism intrinsic to graphene and can it be controllably induced at room temperature? (ii) What are the possible types of ferromagnetism in graphene?

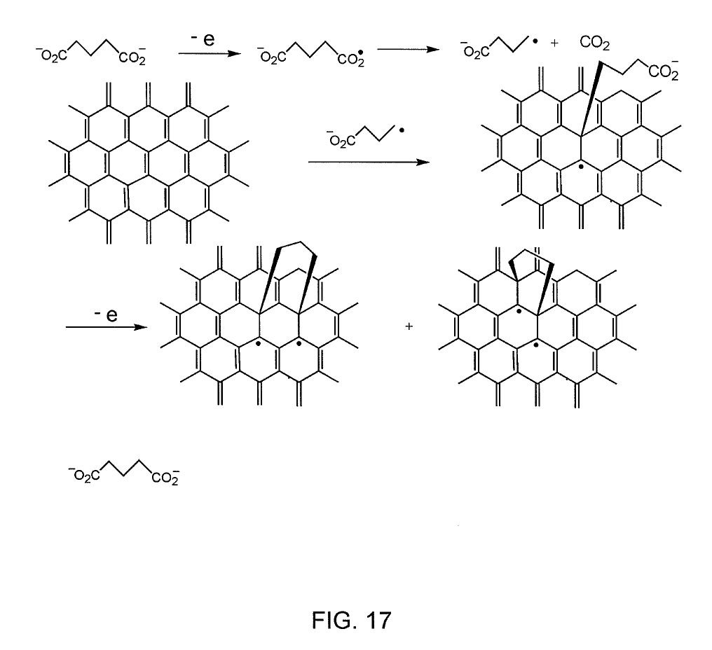

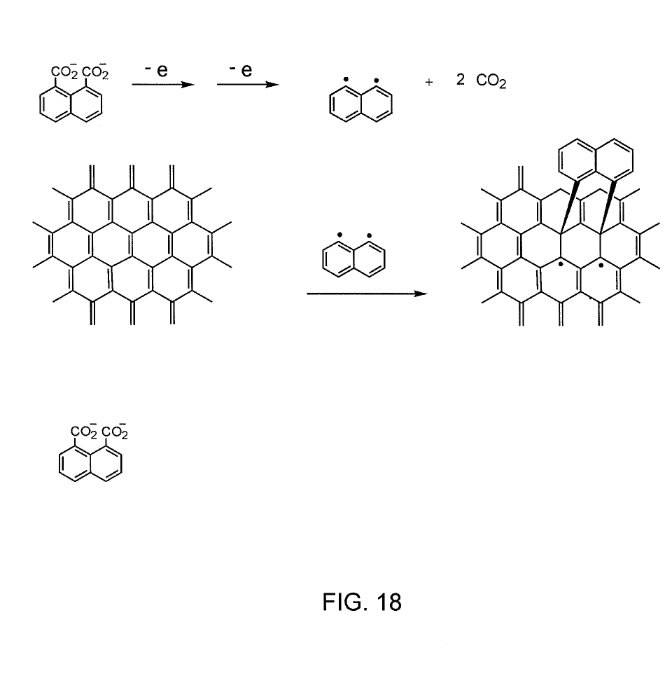

In one aspect of this invention, chemical functionalization is used to controllably induce defects in a manner in which there is a preference for pair-wise functionalization of the A (or B) sub-lattice so that there is an excess of functionalities on one of the two graphene sub-lattices (FIGS. 17 and 18 show specific embodiments of this principle). This guarantees that there will be unpaired spins in the lattice, in some aspects of the invention, and theory has shown that these local moments can couple ferromagnetically to give a magnet. The following set of experiments demonstrates how room temperature ferromagnetism was induced in graphene samples via chemical functionalization. Particularly, an analysis of M-H hysteresis loops and magnetoresistance measured in in-plane and out-of-plane orientations in a temperature range from 2 to 300 K for several samples of graphene before and after functionalization is presented.

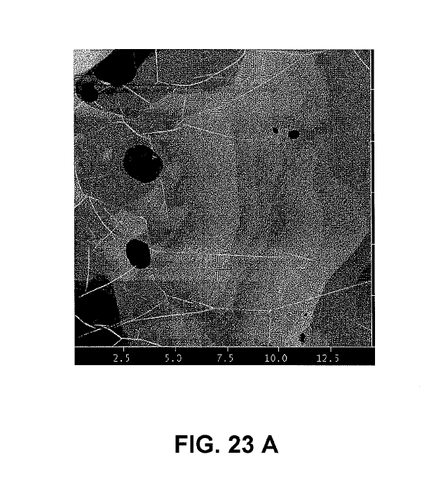

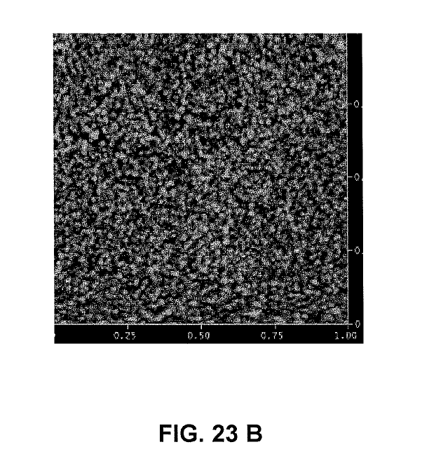

FIGS. 23A-23B show typical atomic force microscopy (AFM) images for a graphene sample before and after functionalization with nitrophenyl, respectively, further referred to as “pristine” and “functionalized” phases, respectively. It should be noted that even the “pristine” phase has long-range defects, i.e. consists of clusters with characteristic lengths of the order of a few microns. In the functionalized case, the characteristic separation between adjacent defect sites is of the order of 0.2 nm. The sample had a rectangular cross-section of approximately 3.5×4.5 mm². The thickness of the seven-layer graphene film was estimated to be approximately 2.35 nm. Only the top layer of the graphene sample was functionalized.

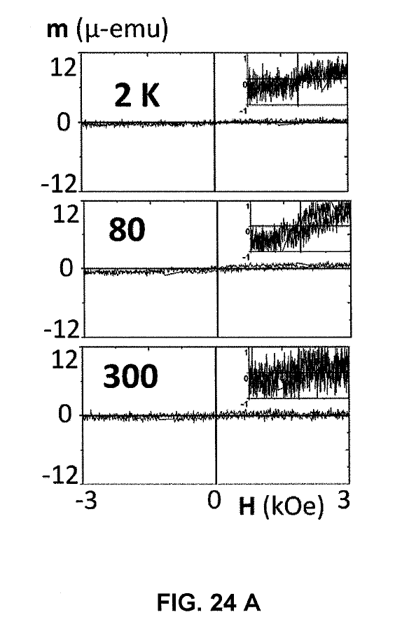

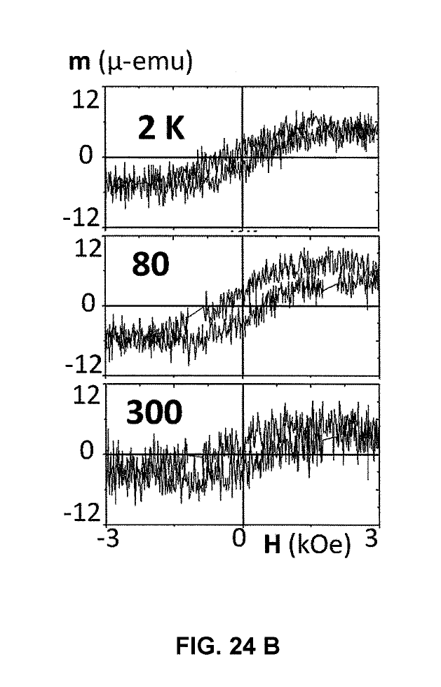

In-plane M-H hysteresis loops at three temperatures—2, 80, and 300 K, respectively, for the above graphene sample at two different phases under study, pristine and functionalized, are shown in FIGS. 24A-24B, respectively. It can be observed that the magnetic signal is substantially increased after functionalization. The increase is by approximately a factor of ten, which made ferromagnetism detectable (above the noise level) at room temperature. Ferromagnetism at room temperature is evident especially for the functionalized phase. In other words, the anticipated Curie temperature for the functionalized phase is above room temperature.

### Vibrating Sample Magnetometry (VSM)

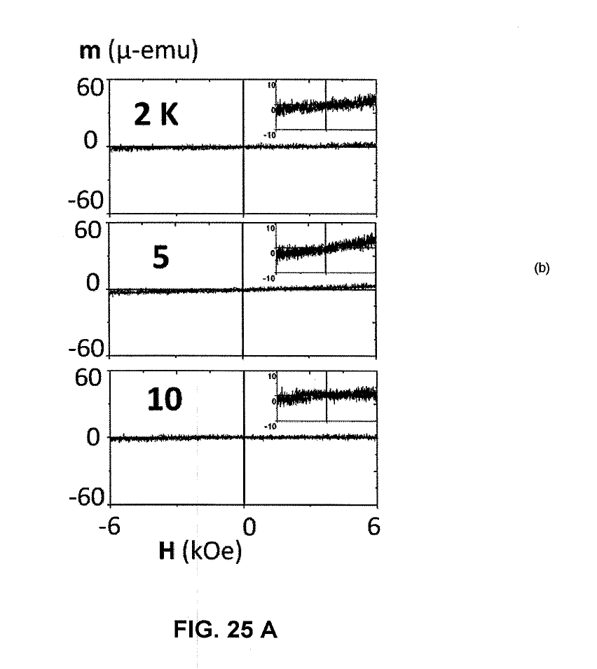

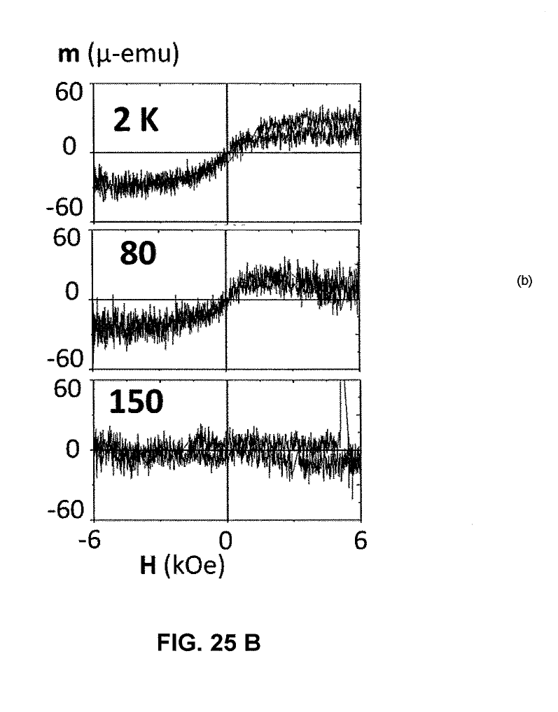

The out-of-plane VSM measurements for the pristine and functionalized phases are shown in FIG. 25A-25B. It can be seen that the pristine phase shows quite negligible magnetism which goes below the noise level between 5 and 10 K. On the contrary, the functionalized phase shows a relatively strong out-of-plane component from 2 to somewhat 100 K. Unlike the in-plane component, the out-of-plane component diminishes below 150 K.

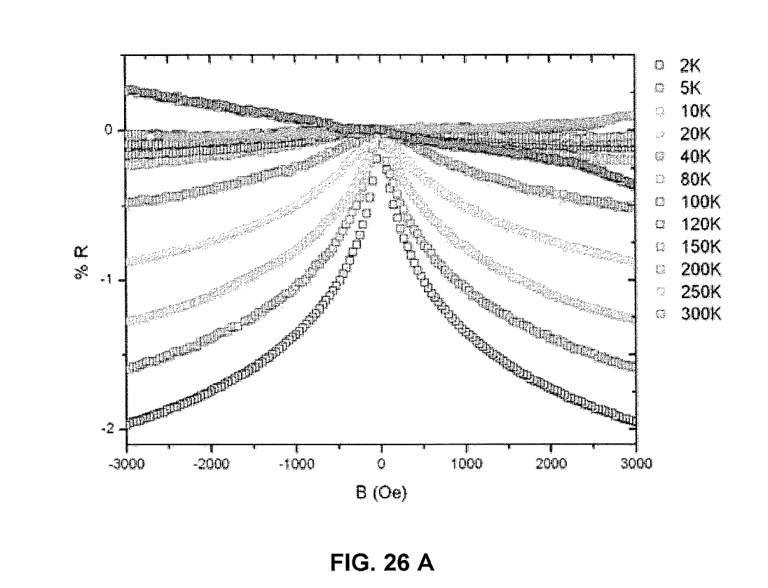

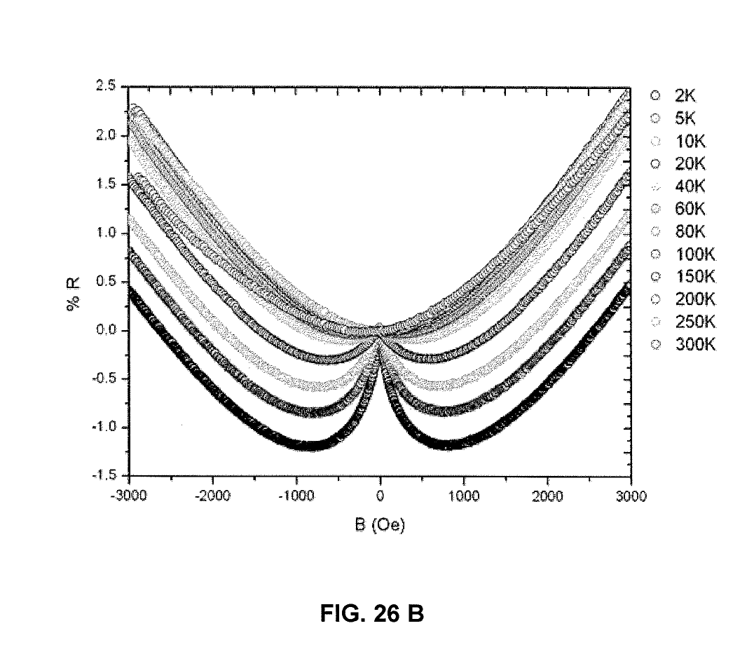

FIGS. 26A-26B show the magnetoresistance (MR) in an out-of-plane direction for a set of temperature values from 2 to 300 K for the pristine and functionalized phases, respectively. There are two distinct and superimposed MR mechanisms, further referred as negative and positive MR, respectively. Each of the two mechanisms is most pronounced in its own field and temperature range. The first mechanism is a negative MR effect with a sharp peak at zero field: the resistance decreases as the magnetic field is increased. The negative MR effect resembles the typical giant MR (GMR) effect as it occurs in granular GMR or layered multilayer structures [P. Grünberg, R. Schreiber, Y. Pang, M. B. Brodsky, and H. Sowers, “Layered magnetic structures: evidence for antiferromagnetic coupling of Fe layers across Cr interlayers,” Phys. Rev. Lett. 57 (19), 2442-5 (1986); M. N. Baibich, J. M. Broto, A. Fert, F. Nguyen Van Dau, F. Petroff, P. Eitenne, G. Creuzet, A. Friederich, and J. Chazelas, “Giant magnetoresistance of (001)Fe/(001)Cr magnetic superlattices,” Phys. Rev. Lett. 61 (21), 2472-5 (1988); A. E. Berkowitz, J. R. Mitchell, M. J. Carey, A. P. Young, S. Zhang, F. E. Spada, F. T. Parker, A. Hutten, and G. Thomas, “Giant magnetoresistance in heterogeneous Cu—Co alloys,” Phys. Rev. Lett. 68 (25), 3745-8 (1992)]. In this case, in the absence of an applied magnetic field, the magnetization in adjacent ferromagnetic grains/clusters or layers is antiparallel due to a weak anti-ferromagnetic coupling. As a magnetic field is applied, the magnetization in the adjacent regions aligns parallel. The resulting reduction of electron scattering is reflected in the observed decrease of the resistance. The above measurements indicate that for the “pristine” graphene sample the negative MR effect is best observed in a relatively low-field field range, from −1000 to +1000 Oe, and at the lowest temperature, 2K, and gradually disappears as the temperature is increased above approximately 100 K. The value of the negative MR is approximately 1% at 2 K. The second mechanism is a positive MR effect, i.e. the resistance increases as an external field is increased. It is displayed in the entire temperature range of interest and most distinguished at room temperature when the negative MR disappears. It could be observed that the effect reminds the magnetoresistance in a semiconductor with a single carrier type for which the resistance is proportional to (1+(μH)²) where μ is the carrier mobility [G. Peleckis, X. L. Wang, S. X. Dou, P. Munroe, J. Ding, B. Lee, “Giant positive magnetoresistance in Fe doped In₂O₃ and InREO₃ (RE=Eu, Nd) composites,” J. Appl. Phys. 103, 07D113 (2008)]. The main difference between the pristine and functionalized phases is the fact that in the functionalized case the negative MR effect is pronounced in a greater field range and at higher temperature values. The noticeable field range of the negative MR effect for the functionalized phase extends substantially above 1 kOe while the characteristic temperature at which the MR changes from negative to positive is above room temperature, compared to less than 80 K for the pristine phase.

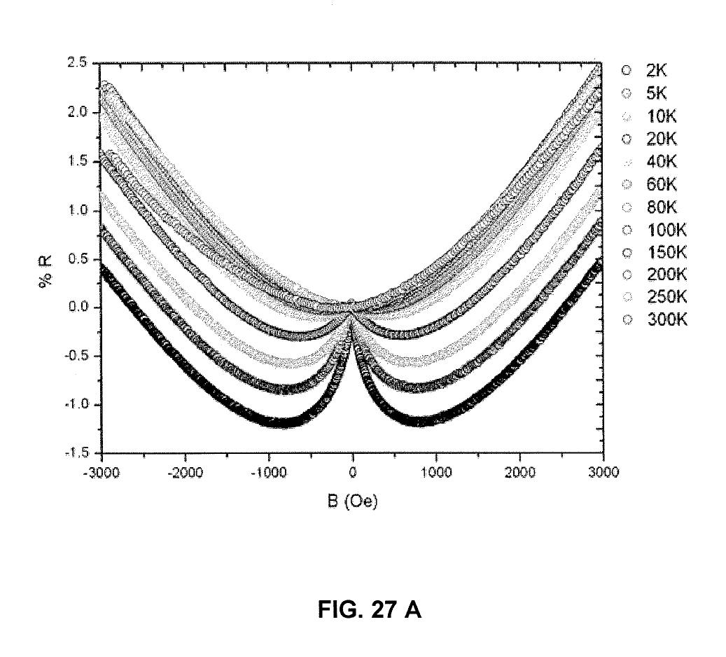

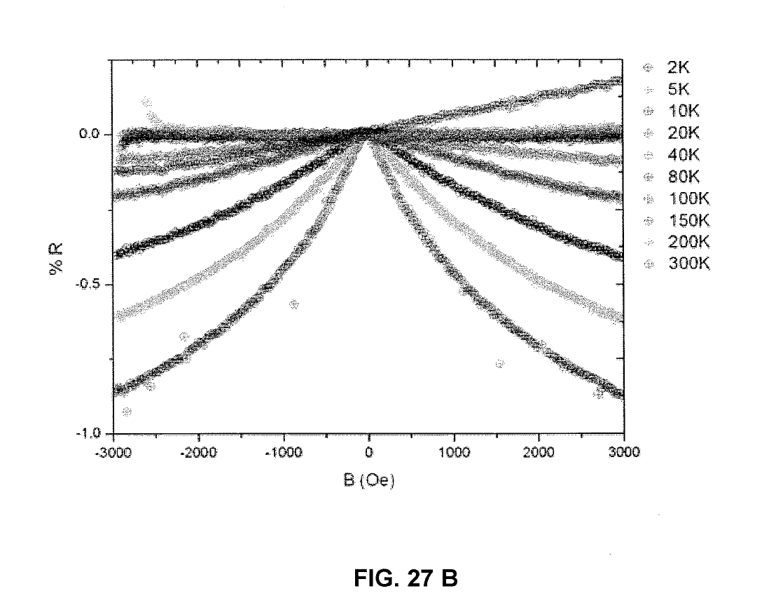

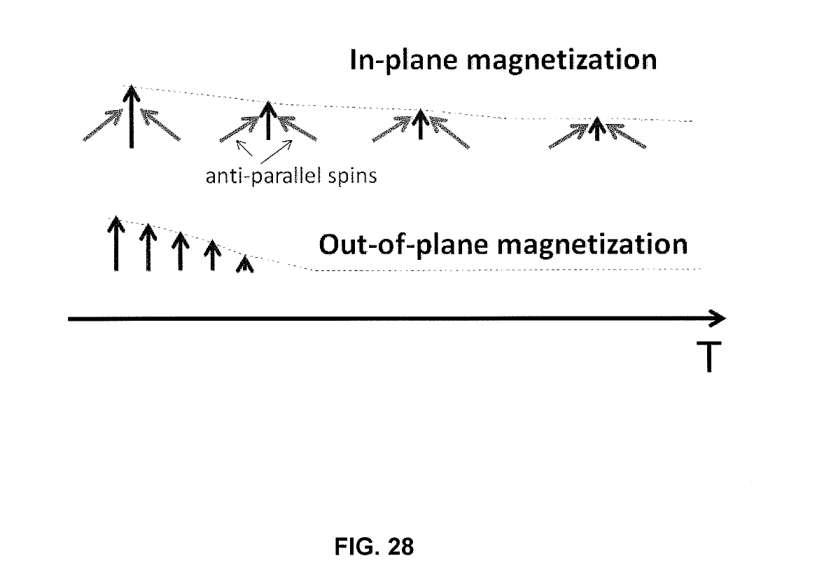

To better understand the anisotropy effects in graphene samples. the magnetoresistance was measured also in an in-plane direction. The out-of-plane and in-plane MR measurements for a set of temperature values from 2 to 300 K for the pristine phase are shown in FIGS. 27A-27B. The key observation is the fact that in the in-plane direction only the negative MR effect is observed. Assuming the origin of the negative MR is similar to that of GMR observed in granular and layered magnetic structures, the characteristic field at which the MR disappears might be attributed to the field necessary to switch coupling between adjacent magnetic sites from anti-parallel to parallel alignment. Putting this together with the earlier discussed results from M-H measurements, we can conclude that the magnetic properties of the sample at room temperature are defined by a mixed state that combines regions with anti-parallel and parallel spin alignments. A schematic illustrating a feasible spin configuration is shown in FIG. 28.

In summary, to demonstrate that chemical functionalization can be used to controllably induce room-temperature ferromagnetism in graphene, correlated vibrating sample magnetometry and magnetoresistance measurements in a temperature range from 2 to 300 K were conducted. The measurements indicated that presence of defects in epitaxially grown graphene sheets indeed could lead to simultaneously observed magnetic and semiconducting properties at room temperature. Though “pristine” samples displayed magnetic properties only in a temperature range below 100 K, after functionalization the same samples became magnetic at least up to room temperature. Both magnetometry and magnetoresistance measurements indicated an anisotropic behavior which might be inherent to the two-dimensional nature of graphene samples.

Although the foregoing description has shown, described, and pointed out the fundamental novel features of the present teachings, it will be understood that various omissions, substitutions, and changes in the form of the detail of the apparatus as illustrated, as well as the uses thereof, may be made by those skilled in the art, without departing from the scope of the present teachings. Consequently, the scope of the present teachings should not be limited to the foregoing discussion.

What is claimed:

1. A modified graphene comprising
at least one sp³ orbital in the modified graphene.

2. The modified graphene of claim 1
wherein the modified graphene is insulating or semiconducting.

3. The modified graphene of claim 1
wherein the modified graphene comprises a local band gap.

4. The modified graphene of claim 1
wherein the modified graphene comprises at least one functional group.

5. The modified graphene of claim 4
wherein the functional group is selected from the group consisting of a substituted or unsubstituted alkyl, alkenyl, alkynyl, heteroalkyl, heteroalkenyl, heteroalkynyl, alkylene, aryl, or heteroaryl group, a heteroatom, and a hydroxyl group.

6. The modified graphene of claim 4
wherein the functional group is phenyl, benzyl, nitrophenyl, nitrobenzyl, naphthyl, dichlorocarbyl, hydroxyl, ketone, or —CF₂(CF₂)ₙCF₃, wherein n is 1-10.

7. The modified graphene of claim 1
wherein the functional group is divalent, and wherein two carbon atoms of the graphene are covalently bonded to the functional group.

8. The modified graphene of claim 1
wherein the modified graphene is modified by removal of a carbon in the graphene backbone.

9. The modified graphene of claim 8
wherein the modified graphene contains a heteroatom or halogen at the site of the removed carbon.

10. The modified graphene of claim 9
wherein the heteroatom is nitrogen or oxygen, or wherein the halogen is fluorine.

11. The modified graphene of claim 1
wherein the modified graphene has a higher resistance than pristine graphene.

12. The modified graphene of claim 1
wherein the modified graphene is saturated to the extent to provide a modified graphene with insulating properties.

13. The modified graphene of claim 1
wherein the modified graphene is saturated to the extent to provide a modified graphene with semiconducting properties.

14. The modified graphene of claim 1
wherein the modified graphene is modified epitaxial graphene.

15. The modified graphene of claim 1,
wherein the modified graphene is partially unsaturated at 2:18 or ˜11% coverage; and
wherein the modified graphene comprises well defined conjugated pathways.

16. The modified graphene of claim 1
wherein the partially unsaturated modified graphene has a lower band gap and higher mobilities than fully saturated modified graphene.

17. The modified graphene of claim 1,
wherein the modified graphene is partially unsaturated at 2:8 or 25% coverage; and
wherein the partially unsaturated modified graphene comprises ill defined conjugated pathways.

18. The modified graphene of claim 7,
wherein the modified graphene has larger band gaps than a partially unsaturated modified graphene having well defined conjugated pathways.

19. A composition comprising the modified graphene of claim 1.

20. The composition of claim 19 further comprising a SiC substrate adjacent the modified graphene.

21. The composition of claim 19 selected from the group consisting of an electronic component or device, a magneto-electronic component or device, a wafer, a ferromagnetic semiconductor, and a field effect transistor (FET).

22. The composition of claim 21 wherein the composition is a wafer and wherein the wafer comprises insulating or semiconducting regions.

23. A method of making the modified graphene of claim 1 comprising
covalently attaching a functional group to a least one carbon atom of a graphene.

24. A method comprising
re-hybridizing the C-atoms in a graphene from sp² to sp³ to form a modified graphene of claim 1.

25. The method of claim 23, further comprising
forming semiconducting or insulating regions on the modified graphene.

26. The method of claim 23
wherein the covalently attaching step further comprises a step selected from the group consisting of adding a dichlorocarbene; spontaneous grafting of an aryl group in a solution of diazonium salts; spontaneous grafting of an aryl group with in-situ generated diazonium salt; and reacting with a radical photochemically generated from an alkyl halide.

27. The method of claim 26 wherein the modified graphene has electronic properties.

28. The method of claim 26 wherein the modified graphene has magnetic properties.

29. The method of claim 23
wherein the covalently attaching step further comprises a step selected from the group consisting of electrochemically attaching an alkyl and/or aryl group to graphene by cyclic voltammetry or electrolysis of carboxylates (the Kolbe reaction); electrochemically attaching an aryl group to graphene by cyclic volammetry scans or electrolysis of a diazonium salt; electrochemically attaching an aryl and/or an alkyl group to graphene by cyclic volammetry scans or electrolysis of an aryl and/or alkyl halide; electrochemically attaching an aryl group to graphene by cyclic volammetry scans or electrolysis of an aryl ketone.

30. The method of claim 29 wherein the modified graphene has electronic properties.

31. The method of claim 29 wherein the modified graphene has magnetic properties.

32. A method of making a patterned graphene comprising
introducing functional groups to graphene to provide semiconducting and or insulating regions of the patterned graphene.

33. The method of claim 32,
wherein the patterned graphene comprises a region, a semiconducting region, and an insulating region.

34. A method comprising
functionalizing graphene to form the modified graphene of claim 1.

35. The method of claim 34
wherein the long-range parallel and/or anti-parallel magnetic order of the graphene samples are created at room temperature.

36. The method of claim 34,
wherein the graphene comprises an A and B lattice; and
wherein said functionalizing step further comprises selectively functionalizing the A or B lattice.

37. A modified graphene produced by the methods of claim 23.

38. A method to control the degree of saturation of modified graphene comprising
selecting a functional group having a size suitable for forming a modified graphene having a preselected degree of saturation; and
functionalizing graphene with the functional group to form the modified graphene having the preselected degree of saturation.

39. The method of claim 38
wherein the functional group modifies the magnetic properties of the modified graphene.

40.-43. (canceled)
